(19) United States
(12) Patent Application Publication
(10) Pub. No.: US 2024/0341641 A1
(43) Pub. Date: Oct. 17, 2024

(54) ENDOSCOPE SYSTEM AND METHOD FOR OPERATING THE SAME

(71) Applicant: FUJIFILM Corporation, Tokyo (JP)

(72) Inventor: Takaaki SAITO, Kanagawa (JP)

(73) Assignee: FUJIFILM Corporation, Tokyo (JP)

(21) Appl. No.: 18/749,529

(22) Filed: Jun. 20, 2024

Related U.S. Application Data
(63) Continuation of application No. PCT/JP2022/037652, filed on Oct. 7, 2022.

(30) Foreign Application Priority Data
Dec. 22, 2021 (JP) 2021-208793
Sep. 20, 2022 (JP) 2022-149521

(51) Int. Cl. A61B 5/1455; A61B 1/00; A61B 1/06; A61B 5/00

(52) U.S. Cl. CPC A61B 5/14552; A61B 1/00045; A61B 1/0638; A61B 1/0646; A61B 5/7246

(57) ABSTRACT

Through a correction value calculation operation, a specific pigment concentration is calculated from each of respective image signals corresponding to a first wavelength range having sensitivity to blood hemoglobin, a second wavelength range different in sensitivity to a specific pigment from the first wavelength range and different in sensitivity to blood hemoglobin from the first wavelength range, a third wavelength range having sensitivity to blood concentration, and a fourth wavelength range having a longer wavelength than the first to third wavelength ranges, and is stored. A representative value is set from a plurality of specific pigment concentrations, an oxygen saturation corrected for the specific pigment is calculated, and an image display is performed using the oxygen saturation.

### CROSS-REFERENCE TO RELATED APPLICATIONS

This application is a Continuation of PCT International Application No. PCT/JP2022/037652 filed on 7 Oct. 2022, which claims priorities under 35 U.S.C § 119 (a) to Japanese Patent Application No. 2021-208793 filed on 22 Dec. 2021, and Japanese Patent Application No. 2022-149521 filed on 20 Sep. 2022. The above application is hereby expressly incorporated by reference, in its entirety, into the present application.

### BACKGROUND OF THE INVENTION

### 1. Field of the Invention

The present invention relates to an endoscope system and a method for operating the same.

### 2. Description of the Related Art

In the recent medical field, oxygen saturation imaging using an endoscope is a technique for calculating the oxygen saturation of blood hemoglobin from a small number of pieces of spectral information of visible light. In the calculation of the oxygen saturation, when a yellow pigment, in addition to blood hemoglobin, is present in the tissue being observed, a spectral signal is affected by the absorption of the pigment, which causes a problem of a deviation of a calculated oxygen saturation value. A technique to address this problem is to perform correction imaging to acquire the spectral characteristics of the tissue being observed before the observation of the oxygen saturation, correct an algorithm for oxygen saturation calculation on the basis of a signal obtained during the imaging, and apply the corrected algorithm to subsequent oxygen saturation calculation (see JP6412252B (corresponding to US2018/0020903A1) and JP6039639B (corresponding to US2015/0238126A1)).

### SUMMARY OF THE INVENTION

In correction for the influence of the absorption of the pigment, which is performed before the calculation of the oxygen saturation, a fixed region of interest is set in an image obtained at the time of correction imaging, and a correction value is calculated on the basis of a representative value such as the average value of pixel values in the fixed region of interest. However, a subtle difference in angle of view or the like at the time of correction imaging may cause the range of an organ appearing in the region of interest to vary each time the imaging is performed. As a result, the correction value that is calculated may also be calculated as a value that is different each time a correction image acquisition operation is performed, and it may be difficult to determine in which operation the value to be employed is calculated.

In the correction performed before the calculation of the oxygen saturation as described above, the calculated oxygen saturation may deviate from the true value if the tissue is observed in a range different from that at the time of the initial correction or if a different tissue is observed.

It is an object of the present invention to provide an endoscope system capable of calculating an accurate oxygen saturation even when the range of an organ appearing in a region of interest includes a plurality of different tissues, and a method for operating the endoscope system.

An endoscope system according to the present invention includes a processor configured to acquire a first image signal from a first wavelength range having sensitivity to blood hemoglobin; acquire a second image signal from a second wavelength range different in sensitivity to a specific pigment from the first wavelength range and different in sensitivity to the blood hemoglobin from the first wavelength range; acquire a third image signal from a third wavelength range having sensitivity to blood concentration; acquire a fourth image signal from a fourth wavelength range having a longer wavelength than the first wavelength range, the second wavelength range, and the third wavelength range; receive an instruction to execute a correction value calculation operation for storing a specific pigment concentration from the first image signal, the second image signal, the third image signal, and the fourth image signal, and store the specific pigment concentration by performing the correction value calculation operation a plurality of times; set a representative value from a plurality of the specific pigment concentrations; calculate an oxygen saturation, based on an arithmetic value acquired from arithmetic processing using the first image signal, the third image signal, and the fourth image signal and based on the representative value; and perform an image display using the oxygen saturation.

Preferably, the processor has a correlation indicating a relationship between the arithmetic value and the oxygen saturation calculated from the arithmetic value, and the processor is configured to correct the correlation, based on at least the representative value.

Preferably, the processor includes a cancellation function of canceling the correction value calculation operation after the correction value calculation operation is performed a plurality of times.

Preferably, the cancellation function is implemented to delete information on an immediately preceding specific pigment concentration or a plurality of the specific pigment concentrations calculated in the correction value calculation operation.

Preferably, the correction value calculation operation stores any number of the specific pigment concentrations in response to a user operation; terminates the correction value calculation operation in response to the user operation or storage of a certain number of the specific pigment concentrations; and calculates the representative value when the correction value calculation operation is terminated.

Preferably, before the correction value calculation operation is performed, a region of interest is set in an image to be captured, and the specific pigment concentration is acquired from an image signal obtained from an image within a range of the region of interest.

Preferably, an upper limit number or a lower limit number of the specific pigment concentrations to be stored in the correction value calculation operation varies in accordance with an area of the region of interest, the upper limit number of the specific pigment concentrations decreases as the area of the region of interest increases, and the lower limit number of the specific pigment concentrations increases as the area of the region of interest decreases.

Preferably, information on the specific pigment concentration is displayed on a screen when the specific pigment concentration is to be stored.

Preferably, in the image display, a region where the oxygen saturation is lower than a specific value is highlighted.

Preferably, the specific pigment is a yellow pigment.

Preferably, the endoscope system includes an endoscope having an imaging sensor provided with a B color filter having a blue transmission range, a G color filter having a green transmission range, and an R color filter having a red transmission range, wherein the first wavelength range is a wavelength range of light transmitted through the B color filter, the second wavelength range is a wavelength range of light transmitted through the B color filter, the second wavelength range is a wavelength range of light having a longer wavelength than the first wavelength range, the third wavelength range is a wavelength range of light transmitted through the G color filter, and the fourth wavelength range is a wavelength range of light transmitted through the R color filter.

Preferably, the blue transmission range is 380 to 560 nm, the green transmission range is 450 to 630 nm, and the red transmission range is 580 to 760 nm.

Preferably, the first wavelength range has a center wavelength of 470±10 nm, the second wavelength range has a center wavelength of 500±10 nm, the third wavelength range has a center wavelength of 540±10 nm, and the fourth wavelength range is a red range.

A method for operating an endoscope system according to the present invention includes a step of acquiring a first image signal from a first wavelength range having sensitivity to blood hemoglobin; a step of acquiring a second image signal from a second wavelength range different in sensitivity to a specific pigment from the first wavelength range and different in sensitivity to the blood hemoglobin from the first wavelength range; a step of acquiring a third image signal from a third wavelength range having sensitivity to blood concentration; a step of acquiring a fourth image signal from a fourth wavelength range having a longer wavelength than the first wavelength range, the second wavelength range, and the third wavelength range; a step of receiving an instruction to execute a correction value calculation operation for storing a specific pigment concentration from the first image signal, the second image signal, the third image signal, and the fourth image signal, and storing the specific pigment concentration by performing the correction value calculation operation a plurality of times; a step of setting a representative value from a plurality of the specific pigment concentrations; a step of calculating an oxygen saturation, based on an arithmetic value acquired from arithmetic processing using the first image signal, the third image signal, and the fourth image signal and based on the representative value; and a step of performing an image display using the oxygen saturation.

According to the present invention, it is possible to calculate an accurate oxygen saturation even when the range of an organ appearing in a region of interest includes a plurality of different tissues.

### BRIEF DESCRIPTION OF THE DRAWINGS

FIG. 1 is an external view of an endoscope system;

FIG. 2 is a block diagram illustrating functions of the endoscope system;

FIG. 3 is a graph illustrating the spectral sensitivity of an imaging sensor;

FIG. 4 is a block diagram illustrating functions of an oxygen saturation image processing unit;

FIG. 5 is a graph illustrating the absorption coefficients of oxyhemoglobin and reduced hemoglobin;

FIG. 6 is a graph illustrating the absorption coefficient of a yellow pigment;

FIGS. 7A to 7C are explanatory diagrams of light emission patterns in an oxygen saturation mode;

FIG. 8 is an explanatory diagram of a second wavelength range of light received by the imaging sensor;

FIG. 9 is an explanatory diagram of emission of illumination light and image signals to be acquired in three types of frames in the oxygen saturation mode;

FIG. 10 is an explanatory diagram of a screen display in a correction value calculation mode;

FIG. 11 is an explanatory diagram of oxygen saturation contours in an XY plane;

FIG. 12 is an explanatory diagram of three types of signal ratios represented in an XYZ space;

FIGS. 13A and 13B are explanatory diagrams of regions of oxygen saturation contours in the XYZ space and regions of oxygen saturation contours in the XY plane;

FIG. 14 is an explanatory diagram of setting of oxygen saturation contours based on an average value of specific pigment concentrations;

FIG. 15 is an explanatory diagram of a screen display in the correction value calculation mode;

FIG. 16 is an explanatory diagram of a correction value calculation operation for acquiring a specific pigment concentration in response to a switch operation;

FIGS. 17A to 17D are explanatory diagrams of patterns of shapes of regions of interest;

FIG. 18 is an explanatory diagram of an operation for canceling an acquired specific pigment concentration;

FIG. 19 is an explanatory diagram of a specific example of calculating an average value of a plurality of specific pigment concentrations to acquire an oxygen saturation;

FIG. 20 is an explanatory diagram of acquisition of a specific pigment concentration from a region of interest;

FIG. 21 is an explanatory diagram of calculation of an oxygen saturation in accordance with an average specific pigment concentration value;

FIG. 22 is an explanatory diagram of a screen display using pseudo-color in an oxygen saturation image;

FIG. 23 is a flowchart illustrating the flow of a series of operations in the oxygen saturation mode;

FIG. 24 is an external view of another example of the endoscope system;

FIG. 25 is an explanatory diagram of light emission control and screen display of another pattern in the oxygen saturation mode;

FIG. 26 is an explanatory diagram of another example of a light source device;

FIG. 27 is a graph illustrating a relationship between a pixel value and reliability;

FIG. 28 is a graph illustrating a relationship between bleeding and reliability;

FIG. 29 is a graph illustrating a relationship between fat, residue, mucus, or residual liquid and reliability;

FIG. 30 is an image diagram of a display that displays a low-reliability region and a high-reliability region having different saturations;

FIG. 31 is an external view of an endoscope system according to a second embodiment;

FIG. 32 is an explanatory diagram of light emission control in a white frame;

FIG. 33 is an explanatory diagram of light emission control in a green frame;

FIG. 34 is an explanatory diagram illustrating functions of a camera head having a color imaging sensor and a monochrome imaging sensor;

FIG. 35 is an explanatory diagram illustrating functions of a dichroic mirror;

FIG. 36 is an explanatory diagram of image signals acquired from light reflected from the white frame;

FIG. 37 is an explanatory diagram of an image signal acquired from transmitted light in the white frame;

FIG. 38 is an explanatory diagram of image signals acquired in the green frame;

FIG. 39 is an explanatory diagram of light emission patterns in the oxygen saturation mode according to the second embodiment;

FIG. 40 is an explanatory diagram illustrating FPGA processing or PC processing;

FIG. 41 is an explanatory diagram illustrating effective pixel data subjected to effective-pixel determination;

FIG. 42 is an explanatory diagram illustrating ROIs;

FIG. 43 is an explanatory diagram illustrating effective pixel data used in the PC processing;

FIG. 44 is an explanatory diagram illustrating reliability calculation, specific pigment concentration calculation, and specific pigment concentration correlation determination;

FIG. 45 is an explanatory diagram illustrating functions of a camera head having four monochrome imaging sensors according to a third embodiment;

FIG. 46 is a graph illustrating emission spectra of violet light and short-wavelength blue light;

FIG. 47 is a graph illustrating an emission spectrum of long-wavelength blue light;

FIG. 48 is a graph illustrating an emission spectrum of green light;

FIG. 49 is a graph illustrating an emission spectrum of red light;

FIG. 50 is a block diagram illustrating functions of a light source device according to a fourth embodiment; and

FIG. 51 is a plan view of a rotary filter.

### DESCRIPTION OF THE PREFERRED EMBODIMENTS

### First Embodiment

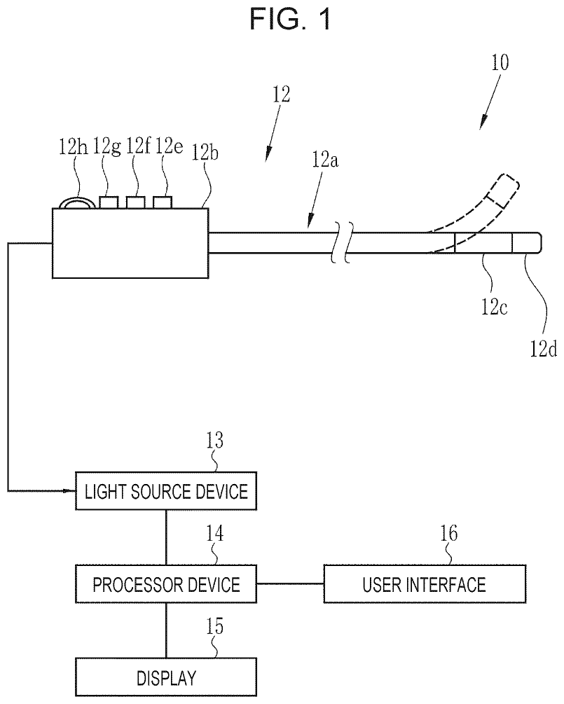

As illustrated in FIG. 1, an endoscope system 10 has an endoscope 12, a light source device 13, a processor device 14, a display 15, and a user interface 16. The endoscope 12 is optically connected to the light source device 13 and is electrically connected to the processor device 14. The light source device 13 supplies illumination light to the endoscope 12.

The endoscope 12 is used to illuminate an observation target with illumination light and perform imaging of the observation target to acquire an endoscopic image. The endoscope 12 has an insertion section 12a to be inserted into the body of the observation target, and an operation section 12b disposed in a proximal end portion of the insertion section 12a. The insertion section 12a is provided with a bending part 12c and a tip part 12d on the distal end side thereof. The bending part 12c is operated by using the operation section 12b to bend in a desired direction. The tip part 12d emits illumination light to the observation target and receives light reflected from the observation target to perform imaging of the observation target. The operation section 12b is provided with a mode switch 12e, which is used for a mode switching operation, a still-image acquisition instruction switch 12f, which is used to provide an instruction to acquire a still image of the observation target, a tissue-color correction switch 12g, which is used for correction during oxygen saturation calculation described below, and a zoom operation unit 12h, which used for a zoom operation.

The processor device 14 is electrically connected to the display 15 and the user interface 16. The processor device 14 receives an image signal from the endoscope 12 and performs various types of processing on the basis of the image signal. The display 15 outputs and displays an image, information, or the like of the observation target processed by the processor device 14. The user interface 16 has a keyboard, a mouse, a touchpad, a microphone, a foot pedal, and the like, and has a function of receiving an input operation such as setting a function.

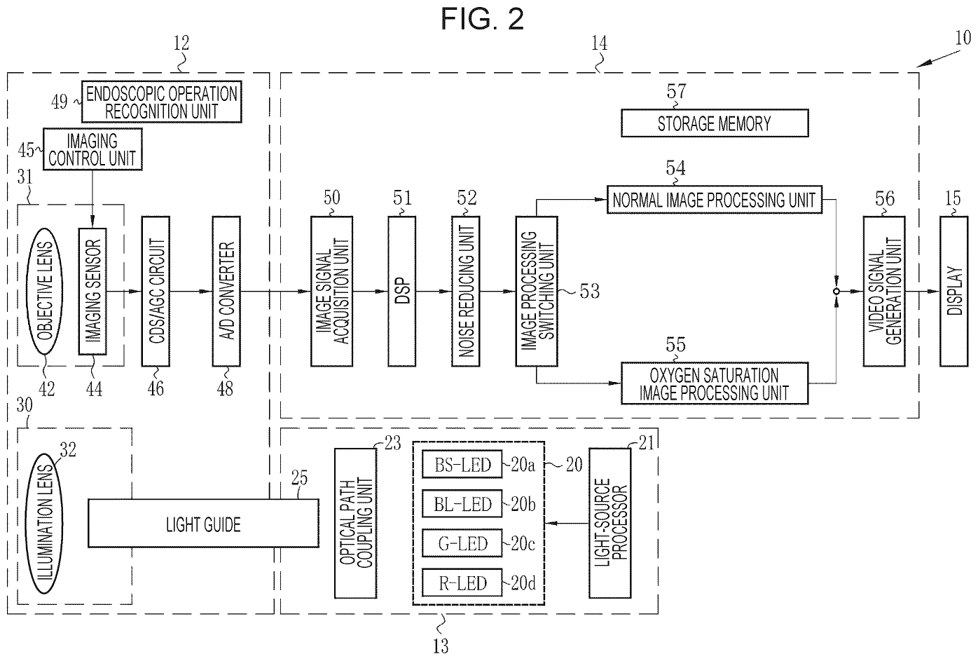

As illustrated in FIG. 2, the light source device 13 includes a light source unit 20 and a light-source processor 21 that controls the light source unit 20. The light source unit 20 has a plurality of semiconductor light sources and turns on or off each of the semiconductor light sources. The light source unit 20 turns on the semiconductor light sources by controlling the amounts of light to be emitted from the respective semiconductor light sources to emit illumination light for illuminating the observation target. In this embodiment, the light source unit 20 has LEDs of four colors, namely, a BS-LED (Blue Short-wavelength Light Emitting Diode) 20a, a BL-LED (Blue Long-wavelength Light Emitting Diode) 20b, a G-LED (Green Light Emitting Diode) 20c, and an R-LED (Red Light Emitting Diode) 20d.

The BS-LED 20a (first semiconductor light source) emits short-wavelength blue light BS of 450 nm±10 nm. The BL-LED 20b (second semiconductor light source) emits long-wavelength blue light BL of 470 nm±10 nm. The G-LED 20c (third semiconductor light source) emits green light G in the green range. The green light G preferably has a center wavelength of 540 nm. The R-LED 20d (fourth semiconductor light source) emits red light R in the red range. The red light R preferably has a center wavelength of 620 nm. The center wavelengths and the peak wavelengths of the LEDs 20a to 20d may be the same or different.

The light-source processor 21 independently inputs control signals to the respective LEDs 20a to 20d to independently control turning on or off of the respective LEDs 20a to 20d, the amounts of light to be emitted at the time of turning on of the respective LEDs 20a to 20d, and so on. The turn-on or turn-off control performed by the light-source processor 21 differs depending on the mode. In a normal mode, the BS-LED 20a, the G-LED 20c, and the R-LED 20d are simultaneously turned on to simultaneously emit the short-wavelength blue light BS, the green light G, and the red light R to perform imaging of a normal image.

The light emitted from each of the LEDs 20a to 20d is incident on a light guide 25 via an optical path coupling unit 23 constituted by a mirror, a lens, and the like. The light guide 25 is incorporated in the endoscope 12 and a universal cord (a cord that connects the endoscope 12 to the light source device 13 and the processor device 14). The light guide 25 propagates the light from the optical path coupling unit 23 to the tip part 12d of the endoscope 12.

The tip part 12d of the endoscope 12 is provided with an illumination optical system 30 and an imaging optical system 31. The illumination optical system 30 has an illumination lens 32. The illumination light propagating through the light guide 25 is applied to the observation target via the illumination lens 32. The imaging optical system 31 has an objective lens 42 and an imaging sensor 44. Light from the observation target irradiated with the illumination light is incident on the imaging sensor 44 via the objective lens 42. As a result, an image of the observation target is formed on the imaging sensor 44.

Driving of the imaging sensor 44 is controlled by an imaging control unit 45. The control of the respective modes, which is performed by the imaging control unit 45, will be described below. A CDS/AGC (Correlated Double Sampling/Automatic Gain Control) circuit 46 performs correlated double sampling (CDS) and automatic gain control (AGC) on an analog image signal obtained from the imaging sensor 44. The image signal having passed through the CDS/AGC circuit 46 is converted into a digital image signal by an A/D (Analog/Digital) converter 48. The digital image signal subjected to A/D conversion is input to the processor device 14. An endoscopic operation recognition unit 49 recognizes a user operation or the like on the mode switch 12e or the tissue-color correction switch 12g included in the operation section 12b of the endoscope 12, and transmits an instruction corresponding to the content of the operation to the endoscope 12 or the processor device 14.

In the processor device 14, a program related to each process is incorporated in a program memory (not illustrated). A central control unit (not illustrated), which is constituted by a processor, executes a program in the program memory to implement the functions of an image signal acquisition unit 50, a DSP (Digital Signal Processor) 51, a noise reducing unit 52, an image processing switching unit 53, a normal image processing unit 54, an oxygen saturation image processing unit 55, a video signal generation unit 56, and a storage memory 57. The video signal generation unit 56 transmits an image signal of an image to be displayed, which is acquired from the normal image processing unit 54 or the oxygen saturation image processing unit 55, to the display 15.

Imaging of the observation target illuminated with the illumination light is implemented using the imaging sensor 44, which is a color imaging sensor. Each pixel of the imaging sensor 44 is provided with any one of a B pixel (blue pixel) having a B (blue) color filter, a G pixel (green pixel) having a G (green) color filter, and an R pixel (red pixel) having an R (red) color filter. For example, the imaging sensor 44 is preferably a color imaging sensor with a Bayer array of B pixels, G pixels, and R pixels, the numbers of which are in the ratio of 1:2:1.

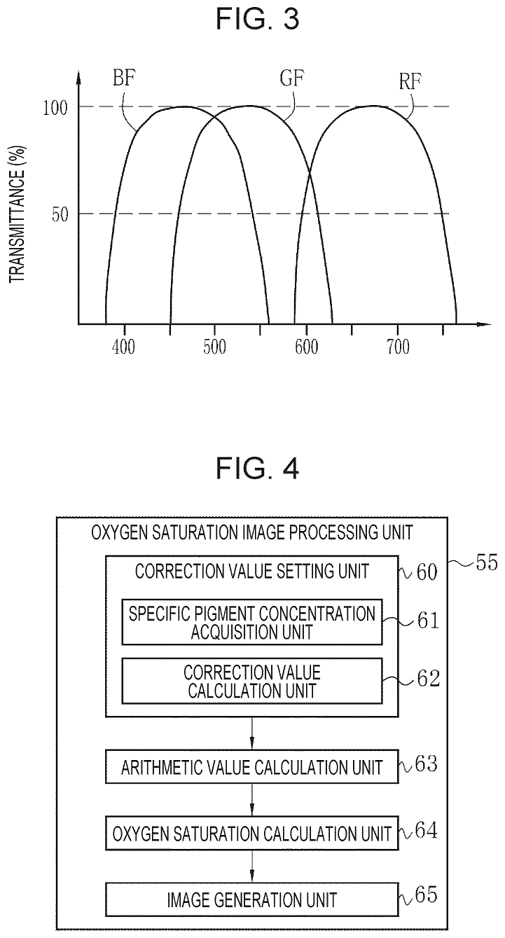

As illustrated in FIG. 3, a B color filter BF mainly transmits light in the blue range, namely, light in the wavelength range of 380 to 560 nm (blue transmission range). A peak wavelength at which the transmittance is maximum appears around 460 to 470 nm. A G color filter GF mainly transmits light in the green range, namely, light in the wavelength range of 450 to 630 nm (green transmission range). An R color filter RF mainly transmits light in the red range, namely, light in the range of 580 to 760 nm (red transmission range).

Examples of the imaging sensor 44 can include a CCD (Charge Coupled Device) imaging sensor and a CMOS (Complementary Metal-Oxide Semiconductor) imaging sensor. Instead of the imaging sensor 44 for primary colors, a complementary color imaging sensor including complementary color filters for C (cyan), M (magenta), Y (yellow), and G (green) may be used. When a complementary color imaging sensor is used, image signals of four colors of CMYG are output. Accordingly, the image signals of the four colors of CMYG are converted into image signals of three colors of RGB by complementary-color-to-primary-color conversion. As a result, image signals of the respective colors of RGB similar to those of the imaging sensor 44 can be obtained.

The image signal acquisition unit 50 receives an image signal input from the endoscope 12, the driving of which is controlled by the imaging control unit 45, and transmits the received image signal to the DSP 51.

The DSP 51 performs various types of signal processing, such as defect correction processing, offset processing, gain correction processing, linear matrix processing, gamma conversion processing, demosaicing processing, and YC conversion processing, on the received image signal. In the defect correction processing, a signal of a defective pixel of the imaging sensor 44 is corrected. In the offset processing, a dark current component is removed from the image signal subjected to the defect correction processing, and an accurate zero level is set. The gain correction processing multiplies the image signal of each color after the offset processing by a specific gain to adjust the signal level of each image signal. After the gain correction processing, the image signal of each color is subjected to linear matrix processing for improving color reproducibility.

Thereafter, gamma conversion processing is performed to adjust the brightness and saturation of each image signal. After the linear matrix processing, the image signal is subjected to demosaicing processing (also referred to as isotropic processing or synchronization processing) to generate a signal of a missing color for each pixel by interpolation. Through the demosaicing processing, all the pixels have signals of RGB colors. The DSP 51 performs YC conversion processing on the respective image signals after the demosaicing processing, and outputs brightness signals Y and color difference signals Cb and Cr to the noise reducing unit 52.

The noise reducing unit 52 performs noise reducing processing on the image signals on which the demosaicing processing or the like has been performed in the DSP 51, by using, for example, a moving average method, a median filter method, or the like. The image signals with reduced noise are input to the image processing switching unit 53.

The image processing switching unit 53 switches the destination to which to transmit the image signals from the noise reducing unit 52 to either the normal image processing unit 54 or the oxygen saturation image processing unit 55 in accordance with the set mode. Specifically, in a case where the normal mode is set, the image processing switching unit 53 inputs the image signals from the noise reducing unit 52 to the normal image processing unit 54. In a case where an oxygen saturation mode is set, the image processing switching unit 53 inputs the image signals from the noise reducing unit 52 to the oxygen saturation image processing unit 55.

The normal image processing unit 54 further performs color conversion processing, such as 3×3 matrix processing, gradation transformation processing, and three-dimensional LUT (Look Up Table) processing, on an Rc image signal, a Gc image signal, and a Bc image signal input for one frame. Then, the normal image processing unit 54 performs various types of color enhancement processing on the RGB image data subjected to the color conversion processing. The normal image processing unit 54 performs structure enhancement processing, such as spatial frequency enhancement, on the RGB image data subjected to the color enhancement processing. The RGB image data subjected to the structure enhancement processing is input to the video signal generation unit 56 as a normal image.

The oxygen saturation image processing unit 55 calculates an oxygen saturation corrected for the tissue color by using image signals obtained in the oxygen saturation mode. A method for calculating the oxygen saturation will be described below. Further, the oxygen saturation image processing unit 55 uses the calculated oxygen saturation to generate an oxygen saturation image in which a low-oxygen region is highlighted by pseudo-color or the like. The oxygen saturation image is input to the video signal generation unit 56. The tissue color correction corrects the influence of specific pigment concentration that is not hemoglobin concentration included in the observation target.

As illustrated in FIG. 4, with the implementation of the function of the oxygen saturation image processing unit 55, the functions of a correction value setting unit 60, an arithmetic value calculation unit 63, an oxygen saturation calculation unit 64, and an image generation unit 65 are implemented. The correction value setting unit 60 has a specific pigment concentration acquisition unit 61 and a correction value calculation unit 62. The oxygen saturation image processing unit 55 is related to the storage memory 57 and the video signal generation unit 56.

The video signal generation unit 56 converts the normal image from the normal image processing unit 54 or the oxygen saturation image from the oxygen saturation image processing unit 55 into a video signal that enables full-color display on the display 15. The video signal after the conversion is input to the display 15. As a result, the normal image or the oxygen saturation image is displayed on the display 15.

The correction value setting unit 60 receives an instruction to execute a correction value calculation operation, which is given by, for example, the user pressing the tissue-color correction switch 12g at any timing, and performs the correction value calculation operation to acquire a specific pigment concentration from the image signals. Preferably, the correction value calculation instruction is given when the observation target is being displayed on a screen. The specific pigment concentration is acquired a plurality of times, and a correction value is calculated. The set specific pigment concentration or correction value is temporarily stored. The storage memory 57 may temporarily store the specific pigment concentration or the correction value.

The specific pigment concentration acquisition unit 61 detects a specific pigment from image signals of a predesignated range of an image being captured, and calculates a specific pigment concentration. The specific pigment concentration acquisition unit 61 has a cancellation function of canceling the correction value calculation operation. The cancellation function receives a cancellation instruction given by, for example, pressing and holding the tissue-color correction switch 12g and executes, for example, deletion of the temporarily stored information on the specific pigment concentration.

The correction value calculation unit 62 calculates a correction value for correcting the influence of the absorption of the specific pigment from a plurality of acquired specific pigment concentrations. A representative value of specific pigment concentrations, which is used to calculate the correction value, is a value determined from a plurality of specific pigment concentrations, and may be a median value, a mode value, or the like rather than an average value. Alternatively, a numerical value summarizing the features of the specific pigment concentrations as a statistic may be used. Performing the correction value calculation operation a plurality of times makes it possible to prevent the use of specific pigment information having a biased value and obtain accurate information on a specific pigment concentration even in a case where a different tissue appears through the correction value calculation operation. The correction value corrects the influence of the specific pigment on the calculation of the oxygen saturation.

Mode switching will be described. The user operates the mode switch 12e to switch the mode setting between the normal mode and the oxygen saturation mode in an endoscopic examination. The destination to which to transmit the image signals from the image processing switching unit 53 is switched in accordance with mode switching.

In the normal mode, the imaging sensor 44 is controlled to capture an image of the observation target being illuminated with the short-wavelength blue light BS, the green light G, and the red light R. As a result, a Bc image signal is output from the B pixels, a Gc image signal is output from the G pixels, and an Rc image signal is output from the R pixels of the imaging sensor 44. These image signals are transmitted to the normal image processing unit 54. The normal image obtained in the normal mode is a white-light-equivalent image obtained by emitting light of three colors, and is different in tint or the like from a white-light image formed by white light obtained by emitting light of four colors.

In the oxygen saturation mode, tissue color correction for performing correction related to a specific pigment by using an image signal is performed to acquire an oxygen saturation image from which the influence of the specific pigment is removed. The oxygen saturation mode further includes a correction value calculation mode for calculating the concentration of a specific pigment and setting a correction value, and an oxygen saturation observation mode for displaying an oxygen saturation image in which the oxygen saturation calculated using the correction value is visualized in pseudo-color or the like. In the correction value calculation mode, an oxygen saturation calculation table is set from the representative value of calculated specific pigment concentrations. In the oxygen saturation mode, three types of frames having different light emission patterns are used to capture images. The oxygen saturation is calculated using an absorption coefficient of blood hemoglobin, which is different for each wavelength range. Blood hemoglobin includes oxyhemoglobin and reduced hemoglobin.

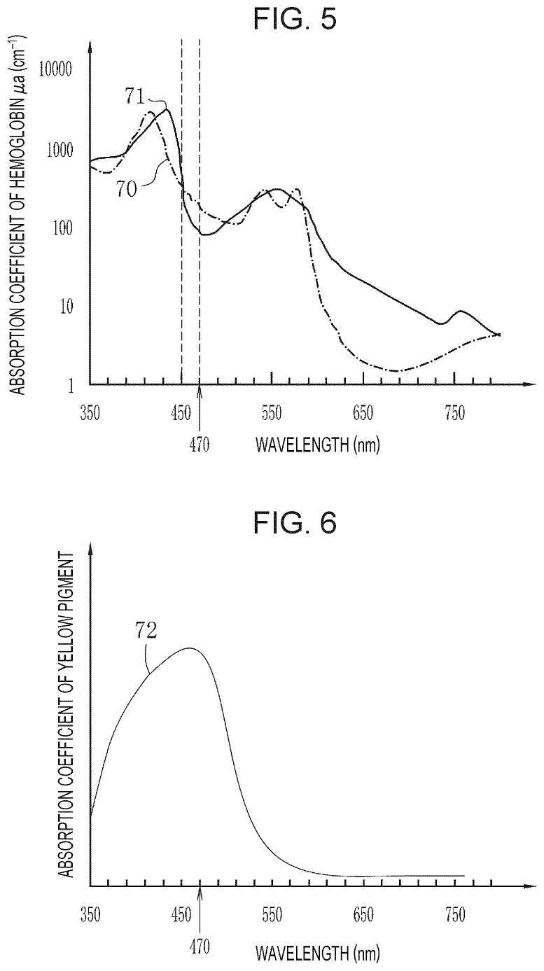

As illustrated in FIG. 5, a curve 70 indicates the absorption coefficient of oxyhemoglobin, and a curve 71 indicates the absorption coefficient of reduced hemoglobin, with the oxygen saturation being closely related to the absorption characteristics of oxyhemoglobin and reduced hemoglobin. For example, in a wavelength range in which the difference in absorption coefficient between oxyhemoglobin and reduced hemoglobin is large, such as a wavelength range around 470 nm, the amount of light absorption changes in accordance with the oxygen saturation of hemoglobin, making it easy to handle information on the oxygen saturation. Accordingly, a B1 image signal corresponding to the long-wavelength blue light BL having a center wavelength of 470±10 nm can be used to calculate the oxygen saturation.

However, an image signal obtained from the long-wavelength blue light BL may be lower than that obtained when a specific pigment other than blood hemoglobin is not included, depending on the presence or absence and concentration of the specific pigment included in the observation target, even if the oxygen saturation is the same, and the calculated oxygen saturation may be apparently shifted to be higher. For example, even if the oxygen saturation can be calculated to be close to 100%, the actual oxygen saturation is about 80%. Examples of the specific pigment include a yellow pigment. The specific pigment concentration refers to the amount of specific pigment present per unit area.

As illustrated in FIG. 6, as indicated by a curve 72, a yellow pigment such as bilirubin included in the observation target has the highest absorption coefficient at a wavelength around 450±10 nm. Accordingly, a wavelength range around 470 nm is a wavelength range in which the amount of light absorption is particularly likely to change in accordance with the concentration of the yellow pigment. The long-wavelength blue light BL is closely related to these absorption characteristics of the yellow pigment. The amount of light absorbed by the yellow pigment is also large at the center wavelengths of 470±10 nm of the long-wavelength blue light BL at which the difference in absorption coefficient between oxyhemoglobin and reduced hemoglobin is large. For this reason, correction is performed to remove the influence of the yellow pigment. The influence of the yellow pigment changes in accordance with the relative relationship with the blood concentration.

The correction is performed using light in a wavelength range in which the absorption coefficients of oxyhemoglobin and reduced hemoglobin have the same value and in which the absorption coefficient of the yellow pigment is larger than those in the other wavelength ranges. That is, it is preferable to use a wavelength range having a center wavelength around 450 nm or 500 nm. An image signal corresponding to a wavelength range around 500 nm is obtained by transmitting the green light G through the B color filter BF.

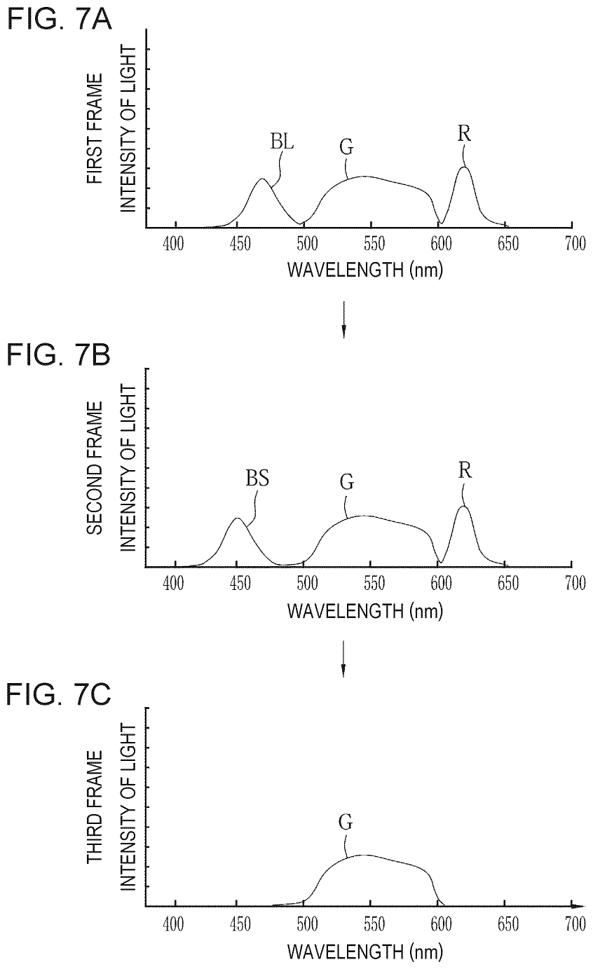

FIGS. 7A to 7C illustrate three types of light emission patterns in the oxygen saturation mode. The light emission patterns illustrated in FIGS. 7A to 7C are switched for each frame to acquire image signals, and the image signals are used to perform correction related to the specific pigment concentration and calculation of the oxygen saturation.

As illustrated in FIG. 7A, in the first frame, the BL-LED 20b, the G-LED 20c, and the R-LED 20d are simultaneously turned on to simultaneously emit the long-wavelength blue light BL, the green light G, and the red light R. A B1 image signal is output from the B pixels, a G1 image signal is output from the G pixels, and an R1 image signal is output from the R pixels.

As illustrated in FIG. 7B, in the second frame, the BS-LED 20a, the G-LED 20c, and the R-LED 20d are simultaneously turned on to simultaneously emit the short-wavelength blue light BS, the green light G, and the red light R. A B2 image signal is output from the B pixels, a G2 image signal is output from the G pixels, and an R2 image signal is output from the R pixels. The second frame has the same light emission pattern as that of light emission in the normal mode. It is preferable that light emission of the G-LED 20c and the R-LED 20d be similar to that in the first frame.

As illustrated in FIG. 7C, in the third frame, the G-LED 20c is turned on to emit the green light G. A B3 image signal is output from the B pixels, a G3 image signal is output from the G pixels, and an R3 image signal is output from the R pixels. Since only the green light G is emitted in the third frame, it is preferable that the G-LED 20c be controlled such that the intensity of the green light G is higher in the third frame than in the first frame and the second frame. The G3 image signal includes image information similar to that of the G2 image signal and is obtained from the green light G having a higher intensity than that in the second frame.

A correction value for correcting the specific pigment is set from, among image signals obtained for three frames in which the observation target is observed, the B1 image signal, the G2 image signal, the R2 image signal, the B3 image signal, and the G3 image signal. The light sources to be turned on in the second frame and the light sources to be turned on in the normal mode have similar configurations.

The B1 image signal (first image signal) includes image information related to a wavelength range (first wavelength range) of light transmitted through the B color filter BF in the long-wavelength blue light BL having a center wavelength of at least 470±10 nm out of the light emitted in the first frame. The first wavelength range is a wavelength range having sensitivity to the specific pigment concentration other than that of blood hemoglobin among pigments included in the observation target and to blood hemoglobin.

The B3 image signal (second image signal) includes image information related to a wavelength range (second wavelength range) of light transmitted through the B color filter BF in the green light G emitted in the third frame. The second wavelength range is a wavelength range different in sensitivity to the specific pigment from the first wavelength range and different in sensitivity to blood hemoglobin from the first wavelength range.

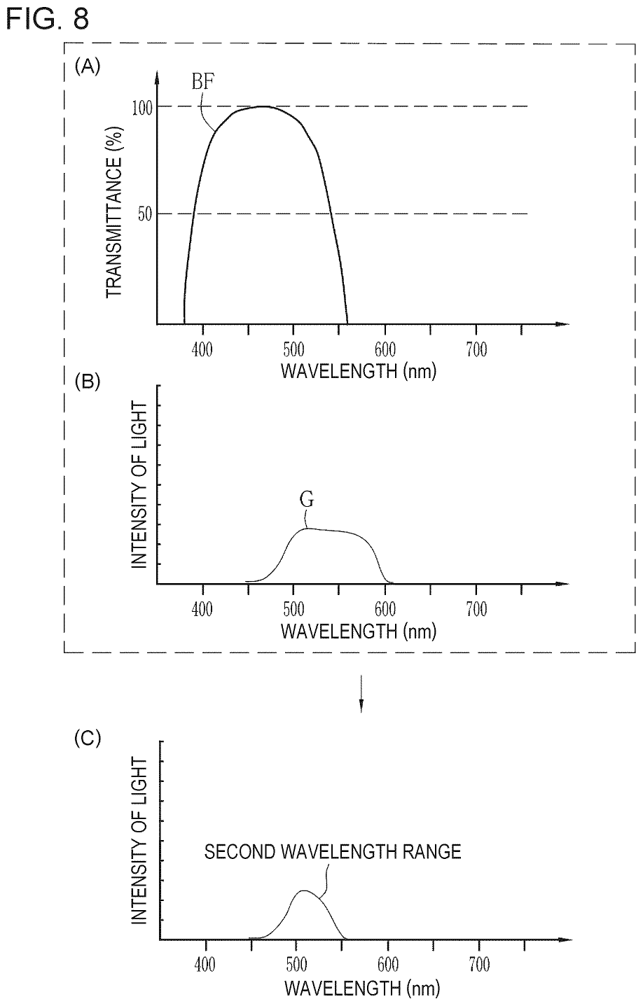

The second wavelength range illustrated in FIG. 8 is obtained by transmitting the green light G having a wavelength range around 470 to 600 nm illustrated in part (B) of FIG. 8 through the B color filter BF that transmits light having a wavelength range of 380 to 560 nm illustrated in part (A) of FIG. 8. The B pixels receive light in a wavelength range around 470 nm to 560 nm. The B color filter BF has a peak transmittance at 450 nm, with the transmittance decreasing toward the long-wavelength side. The intensity of the green light G decreases toward the wavelength side shorter than a center wavelength of 540±10 nm. For this reason, as illustrated in part (C) of FIG. 8, the second wavelength range has a center wavelength of 500±10 nm.

The G2 image signal (third image signal) includes image information related to a wavelength range (third wavelength range) of light transmitted through the G color filter GF in at least the green light G out of the light emitted in the second frame. The third wavelength range is a wavelength range having sensitivity to blood concentration. In addition, like the G2 image signal, the G3 image signal includes image information related to the third wavelength range, and thus can be used as a third image signal for a correction value calculation operation.

The R2 image signal (fourth image signal) includes image information related to a wavelength range (fourth wavelength range) of light transmitted through the R color filter RF in at least the red light R out of the light emitted in the second frame. The fourth wavelength range is a red range having a longer wavelength than the first wavelength range, the second wavelength range, and the third wavelength range, and has a center wavelength of 620±10 nm.

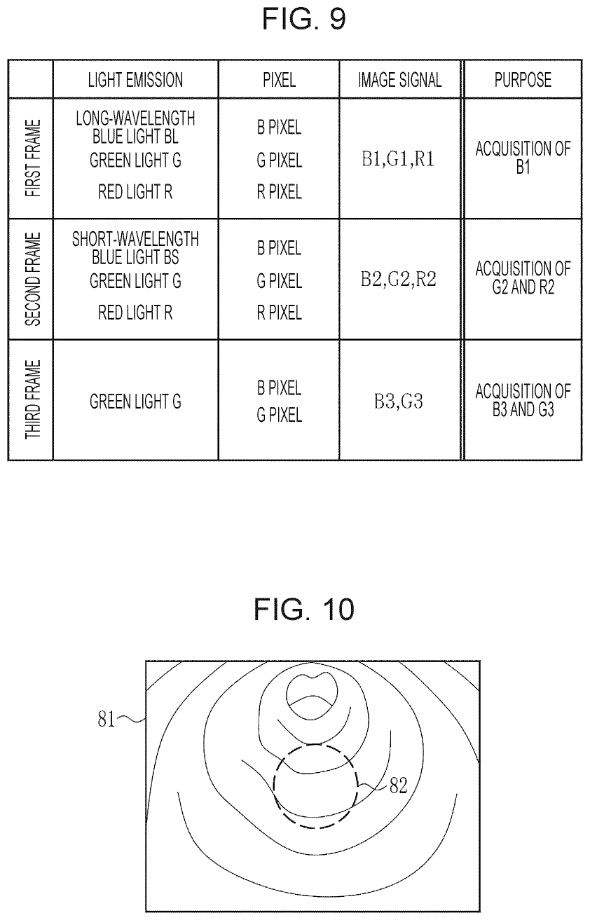

As illustrated in FIG. 9, in the oxygen saturation mode, the B1 image signal, the G2 image signal, the R2 image signal, the B3 image signal, and the G3 image signal are acquired from the first to third frames, and the oxygen saturation corrected for the specific pigment is calculated.

In the correction value calculation mode, a correction value is set using image signals acquired by observing the observation target. The image signals include a first image signal acquired from the first wavelength range having sensitivity to the specific pigment concentration other than that of blood hemoglobin among pigments included in the observation target and to blood hemoglobin, a second image signal acquired from the second wavelength range different in sensitivity to the specific pigment from the first wavelength range and different in sensitivity to blood hemoglobin from the first wavelength range, a third image signal acquired from the third wavelength range having sensitivity to blood concentration, and a fourth image signal acquired from the fourth wavelength range having longer wavelengths than the first wavelength range, the second wavelength range, and the third wavelength range.

In the correction value calculation mode, an instruction for executing a correction value calculation operation for performing correction on the specific pigment, which is given by a user operation or the like, is received. In the correction value calculation operation, specific pigment concentrations are calculated from the first image signal, the second image signal, the third image signal, and the fourth image signal and are stored. A correction value is set from the representative value of the plurality of specific pigment concentrations stored by performing the correction value calculation operation a plurality of times.

After the correction value is set, the correction value calculation mode is switched to the oxygen saturation observation mode, and arithmetic values are acquired from arithmetic processing using the first image signal, the third image signal, and the fourth image signal. The oxygen saturation is calculated from the arithmetic values on the basis of the correction value, and image display using the oxygen saturation is performed. In the image display, a region with low oxygen saturation is preferably highlighted.

The arithmetic value calculation unit 63 calculates arithmetic values by arithmetic processing based on the first image signal, the third image signal, and the fourth image signal. The first image signal is highly dependent on not only the oxygen saturation but also the blood concentration. Accordingly, the first image signal is compared with the fourth image signal having low blood concentration dependence to calculate the oxygen saturation. The third image signal also has blood concentration dependence. The difference in blood concentration dependence among the first image signal, the fourth image signal, and the third image signal is used, and the third image signal is used as a reference image signal (normalized image signal).

Specifically, the arithmetic value calculation unit 63 calculates, as arithmetic values to be used for the calculation of the oxygen saturation, a signal ratio B1/G2 between the B1 image signal and the G2 image signal and a signal ratio R2/G2 between the R2 image signal and the G2 image signal and uses a correlation therebetween to accurately determine the oxygen saturation without being affected by the blood concentration. The signal ratio B1/G2 and the signal ratio R2/G2 are each preferably converted into a logarithm (In). Alternatively, color difference signals Cr and Cb, or a saturation S, a hue H, or the like calculated from the B1 image signal, the G2 image signal, and the R2 image signal may be used as the arithmetic values.

As illustrated in FIG. 10, image signals to be used to calculate the correction value are acquired from a region surrounded by a region of interest 82 in an image display region 81 displayed on the display 15 in the correction value calculation mode. The region of interest 82 is preferably set in advance at least before a correction value calculation operation described below and constantly displayed in the correction value calculation mode. A specific pigment concentration is acquired using the image signals acquired from the region of interest 82. A fixed correction value is set from a representative value such as an average value of specific pigment concentrations acquired a plurality of times. As a result, a variation in the correction value each time a specific pigment concentration is acquired, which is caused by a difference in angle of view or the like, is reduced, and the same correction is performed to reduce the burden of correction value calculation, enabling stable calculation of the oxygen saturation.

The oxygen saturation calculation unit 64 refers to the oxygen saturation calculation table and applies the arithmetic values calculated by the arithmetic value calculation unit 63 to oxygen saturation contours to calculate the oxygen saturation. The oxygen saturation contours are contours formed substantially along the horizontal axis direction, each of the contours being obtained by connecting portions having the same oxygen saturation. The contours with higher oxygen saturations are located on the lower side in the vertical axis direction. For example, the contour with an oxygen saturation of 100% is located below the contour with an oxygen saturation of 80%.

For the oxygen saturation, an oxygen saturation calculation table generated in advance by simulation, a phantom, or the like is referred to, and arithmetic values are applied to the oxygen saturation contours. In the oxygen saturation calculation table, correlations between oxygen saturations and arithmetic values constituted by the signal ratio B1/G2 and the signal ratio R2/G2 in an XY plane (two-dimensional space) formed by a Y-axis Ln (B1/G2) and an X-axis Ln (R2/G2) are stored as oxygen saturation contours. Each signal ratio is preferably converted into a logarithm (In).

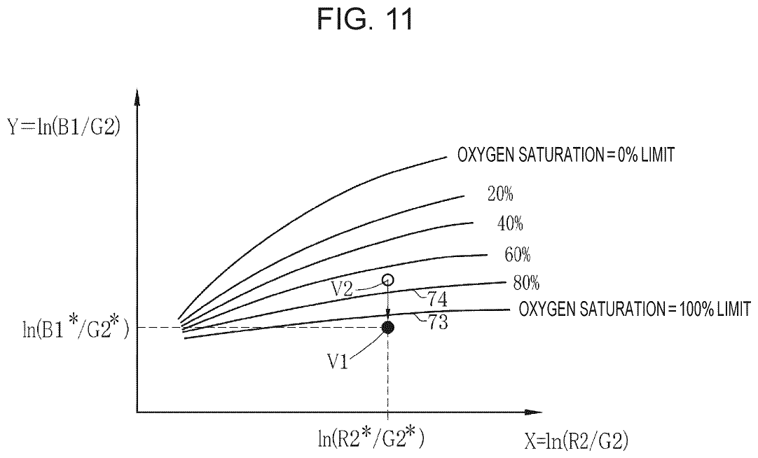

FIG. 11 illustrates an arithmetic value V1 and an arithmetic value V2 for the same observation target, the arithmetic value V1 being applied to the oxygen saturation contour without being corrected, the arithmetic value V2 being corrected for the specific pigment and then applied to the oxygen saturation contours obtained from the oxygen saturation calculation table. Since the B1 image signal is a lower signal for a higher specific pigment concentration, the value of the signal ratio B1/G2 shifts downward, resulting in an increase in apparent oxygen saturation. The uncorrected arithmetic value V1 is located below a contour 73 with an oxygen saturation of 100%, whereas the corrected arithmetic value V2 is located above a contour 74 with an oxygen saturation of 80%. Since the R2 image signal is less affected by the specific pigment, the apparent values of the arithmetic values in the X-axis direction do not substantially change. The correction corrects a shift of the arithmetic values to values lower than the values otherwise on the Y-axis with respect to the oxygen saturation contours due to the influence of the specific pigment. The specific pigment concentration for the arithmetic value V1 is represented by CP, and the specific pigment concentration for the arithmetic value V2 is 0 or a negligible value.

The specific pigment concentration acquisition unit 61 calculates a specific pigment concentration on the basis of the first to fourth image signals. Specifically, in the calculation of the oxygen saturation, the influence of the specific pigment concentration is corrected by using three types of signal ratios, namely, a signal ratio B3/G3 in addition to the correlation between the signal ratio B1/G2 and the signal ratio R2/G2. Since the emission of the green light G in the third frame is different from that in the first frame and the second frame, the G3 image signal is preferably used as the reference image signal for the B3 image signal.

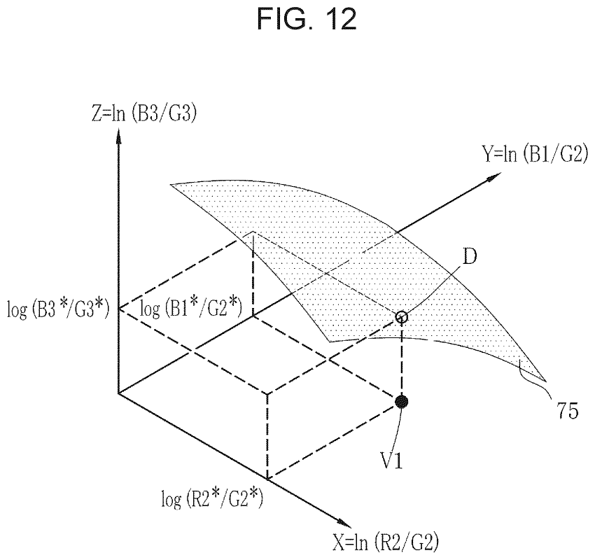

As illustrated in FIG. 12, for the oxygen saturation contours, a Z-axis using the signal ratio B3/G3 can be added to the correlations using the X-axis represented by the signal ratio R2/G2 and the Y-axis represented by the signal ratio B1/G2, and the three types of signal ratios are represented by an XYZ space. The XYZ space can represent a correlation related to the oxygen saturation, the blood concentration, and the specific pigment, which is determined in advance by simulation, a phantom, or the like. The correlation can be represented by a visualized region, which is a curved surface on which oxygen saturation contours are present under a condition where the specific pigment concentration is constant. A region 75 for the specific pigment concentration CP will be described. The arithmetic value V1 on the XY plane is set as three-dimensional coordinates D also having a value including the Z-axis, thereby making it possible to determine an accurate oxygen saturation. In the XYZ space, a corresponding curved surface is set for each set of three-dimensional coordinates, thereby making it possible to calculate the oxygen saturation in accordance with the specific pigment concentration.

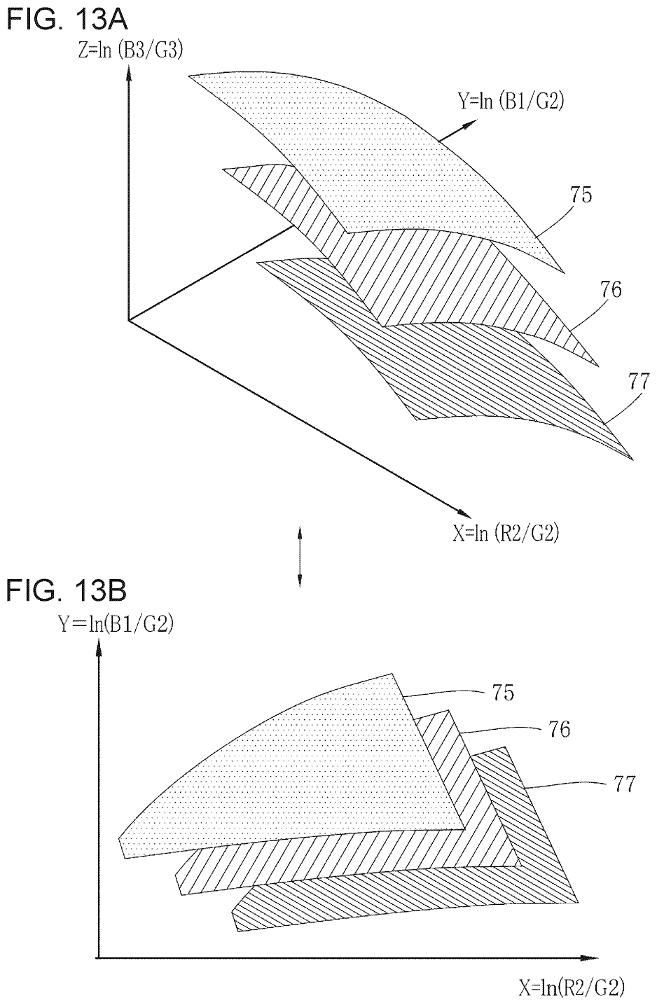

As illustrated in FIG. 13A, curved surfaces in the XYZ space and ranges of contours in the XY plane are set for the respective specific pigment concentrations. The specific pigment concentrations, which are represented by CP, CQ, and CR in order from lowest to highest, will be described. As indicated by the region 75 corresponding to the specific pigment concentration CP, a region 76 corresponding to the specific pigment concentration CQ, and a region 77 corresponding to the specific pigment concentration CR, as the specific pigment concentration increases in the XYZ space, the region shifts toward larger values in the X-axis direction and shifts toward smaller values in the Y-axis direction and the Z-axis direction. As illustrated in FIG. 13B, the regions of the oxygen saturation contours represented by the curved surfaces in the XYZ space are converted and represented by XY planes for the respective specific pigment concentrations. In the XY planes, as the specific pigment concentration increases, the region increases in the X-axis direction and decreases in the Y-axis direction. That is, a shift is made in the lower right direction.

Since the regions of the oxygen saturation contours are determined from the correlation using the specific pigment concentrations, the correlation of the three types of signal ratios can be fixed for the same observation target that can be determined to have approximately the same specific pigment concentration, and the positions of the oxygen saturation contours in the XY planes can be determined. The amount of movement of a region with respect to the region in the reference state where the specific pigment concentration is 0 or a negligible value is a correction value. That is, the amount of movement from the region with a specific pigment concentration of 0 to the region with the specific pigment concentration CP is a correction value for the specific pigment concentration CP.

In a reference state where the correlation indicating the relationship between an arithmetic value and an oxygen saturation calculated from the arithmetic value is not affected by the specific pigment concentration, correction related to the specific pigment concentration is received. The correlation in the reference state is corrected to a correlation corresponding to the specific pigment concentration on the basis of at least a representative value such as the average value of the specific pigment concentrations calculated in accordance with the correction value calculation operation. The following describes a case where the correlation varies from the reference state due to correction based on the average value of the calculated specific pigment concentrations.

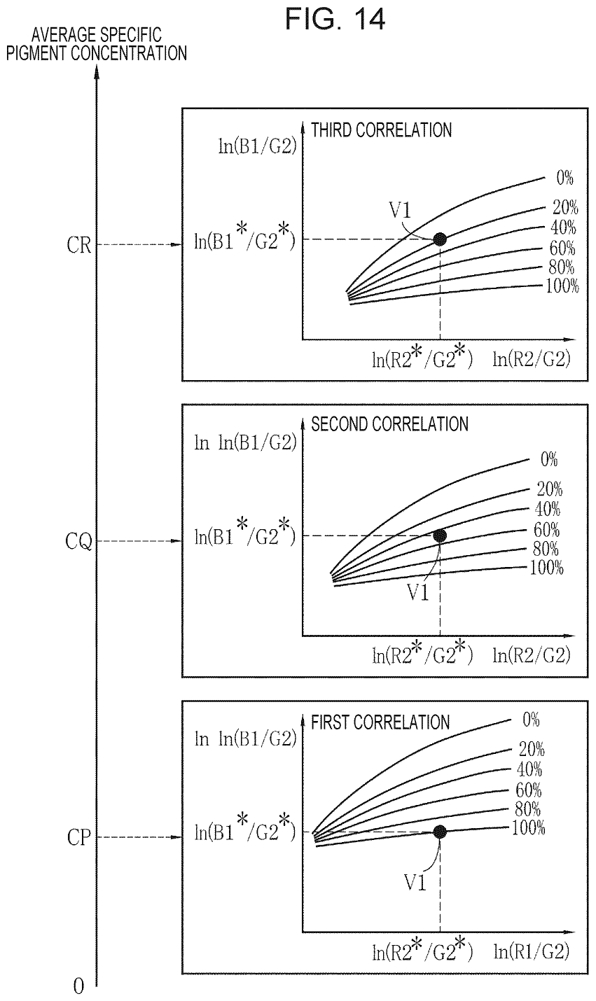

Three-stage patterns in which, as illustrated in FIG. 14, an average specific pigment concentration value CA has values CP, CQ, and CR in order from lowest to highest will be described. The oxygen saturation contours set in accordance with the specific pigment concentration values vary in correlation such as position from the reference state where the specific pigment concentration is 0 or a negligible value. When the average specific pigment concentration value CA has the value CP, the correlation with the position of the oxygen saturation contour is changed to a first correlation. In a second correlation when the average specific pigment concentration value CA has the value CQ, the oxygen saturation contour is entirely lower than that in the first correlation, and the oxygen saturation at the same arithmetic value V1 is lower. In a third correlation when the average specific pigment concentration value CA has the value CR, the oxygen saturation contour is entirely lower than that in the second correlation, and the oxygen saturation at the same arithmetic value V1 is lower.

When the concentration of the specific pigment in the image is higher, that is, the average specific pigment concentration value CA has a larger value, the oxygen saturation contour obtained from the oxygen saturation calculation table is entirely lower for the signal ratio B1/G2 along the Y-axis, resulting in a lower oxygen saturation for the same arithmetic value. Accordingly, a correlation corresponding to the average specific pigment concentration value CA is applied to an arithmetic value obtained from the signal ratio B1/G2 and the signal ratio R2/G2 to perform correction related to the specific pigment, thereby making it possible to apply the arithmetic value to calculate the oxygen saturation. The correction for the influence of the specific pigment concentration is correction for the relative positions of the arithmetic value and the oxygen saturation contour. For this reason, instead of the amount of movement by which the oxygen saturation contour is shifted from the reference state where the specific pigment concentration is 0 or a negligible value, the arithmetic value may be corrected to shift the oxygen saturation contour.

The signal ratio B1/G2 and the signal ratio R2/G2 are rarely extremely large or extremely small. That is, combinations of the values of the signal ratio B1/G2 and the signal ratio R2/G2 are rarely distributed below the upper-limit contour with an oxygen saturation of 100% or, conversely, are rarely distributed above the lower-limit contour with an oxygen saturation of 0%. If the combinations are distributed below the upper-limit contour, the oxygen saturation calculation unit 64 sets the oxygen saturation to 100%. If the combinations are distributed above the lower-limit contour, the oxygen saturation calculation unit 64 sets the oxygen saturation to 0%. If no points corresponding to the signal ratio B1/G2 and the signal ratio R2/G2 are distributed between the upper-limit contour and the lower-limit contour, a display may be provided to indicate that the reliability of the oxygen saturation for the corresponding pixel is low, and the oxygen saturation is not calculated.

A correction value calculation operation and a correction value confirmation operation in the correction value calculation mode will be described. In the correction value calculation operation, the specific pigment concentration can be calculated by applying the acquired three types of signal ratios to the regions of the oxygen saturation contours in the XYZ space described above. That is, the amount of movement between the region of the oxygen saturation contour at the reference position and the region of the oxygen saturation contour corrected for the specific pigment concentration when the specific pigment concentration is 0 or negligible is obtained. In addition, information on the specific pigment concentrations is displayed on the display 15, thereby making it possible to compare the specific pigment concentrations and check that the same observation condition and observation target are used when calculating a plurality of correction values.

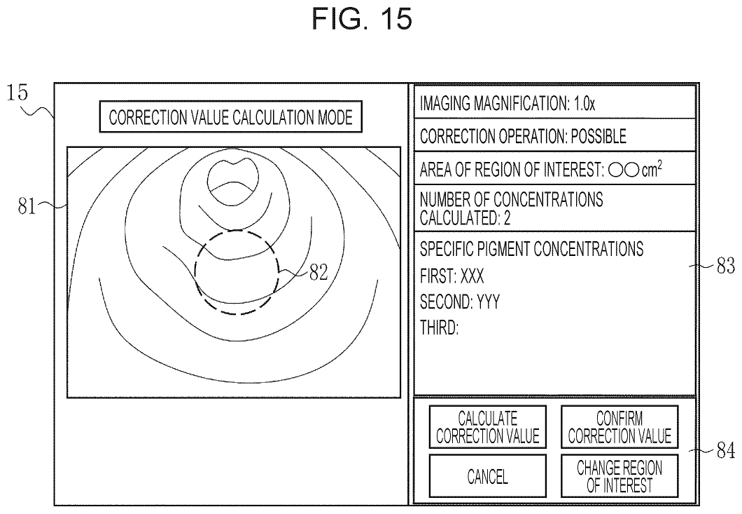

As illustrated in FIG. 15, in the correction value calculation mode, the display 15 displays the image display region 81, the region of interest 82, an image information display region 83, and a command region 84. The image display region 81 displays an image captured with an endoscope, and the region of interest 82 is provided at a designated position in the image, such as the center. In the region of interest 82, a range in which specific pigment concentrations are to be calculated in the correction value calculation mode is indicated with a circle or any other shape. The image information display region 83 displays imaging information such as an imaging magnification, the area of the region of interest 82, the values of the calculated specific pigment concentrations, and so on. The command region 84 indicates a command executable in accordance with a user instruction in the correction value calculation mode. Examples of the command include a concentration acquisition operation, a cancellation operation, a correction value confirmation operation, and a region-of-interest change operation.

In the correction value calculation operation, a site or an organ for which the oxygen saturation is to be measured is depicted in the region of interest 82 in the correction value calculation mode, and specific pigment concentrations are acquired. The region of interest 82 is set in an image to be captured before the correction value calculation operation is performed, and the correction value calculation operation is performed to acquire the specific pigment concentrations from the three types of signal ratios within the range of the region of interest 82. The cancellation function for canceling the correction value calculation operation executes cancellation in accordance with a cancellation instruction given by the user when, for example, the region of interest 82 erroneously includes an inappropriate portion.

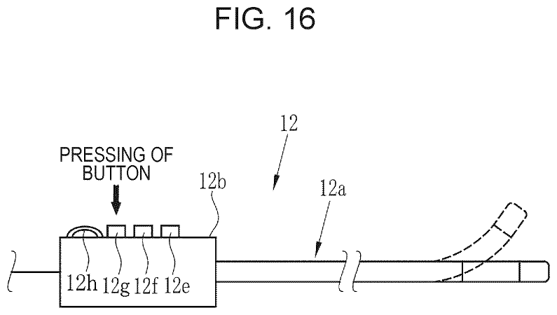

As illustrated in FIG. 16, the user presses the tissue-color correction switch 12g, which is included in the operation section 12b of the endoscope 12 to issue an instruction necessary for tissue color correction, such as a correction value calculation instruction, a correction value confirmation instruction, or a cancellation instruction. In the correction value calculation mode, the mode switch 12e can be used to switch between the oxygen saturation mode and the normal mode, the still-image acquisition instruction switch 12f can be used to acquire a captured image, and the zoom operation unit 12h can be used to perform an operation of enlarging or shrinking the image display region 81 or the region of interest 82. During the operation of the tissue-color correction switch 12g, an instruction corresponding to the number of times the tissue-color correction switch 12g is pressed by the user in a certain period of time or the number of seconds over which the tissue-color correction switch 12g is pressed is issued to the oxygen saturation image processing unit 55. For example, a single press of the tissue-color correction switch 12g provides a correction value calculation instruction, two presses of the tissue-color correction switch 12g provide a correction value confirmation instruction, and a long press of the tissue-color correction switch 12g provides a cancellation instruction.

In response to the correction value calculation instruction, a correction value calculation operation is performed to calculate a specific pigment concentration from an image signal of a range surrounded by the region of interest 82 and temporarily store the specific pigment concentration in the storage memory 57. The correction value calculation operation is performed using not a single specific pigment concentration but an average value of a plurality of specific pigment concentrations, thereby making it possible to increase the accuracy of the correction value. In response to the correction value confirmation instruction, a representative value of specific pigment concentrations is calculated and a correction value is set. Preferably, the number of time the correction value calculation operation is to be performed varies in accordance with the area of the region of interest 82 set in the image display region 81. If the acquisition of specific pigment concentrations or the calculation of a representative value of specific pigment concentrations fails to be performed appropriately, a cancellation instruction is preferably issued to cancel or redo the operation. In the storage of the specific pigment concentrations, information on the three types of signal ratios corresponding to the specific pigment concentrations is also stored as information on the specific pigment concentrations.

Instead of an instruction using the tissue-color correction switch 12g or as selective use of the content of the instruction, any one of foot-pedal input, audio input, and keyboard or mouse operation may be used. Alternatively, the instruction may be given by selecting a command displayed in the command region 84.

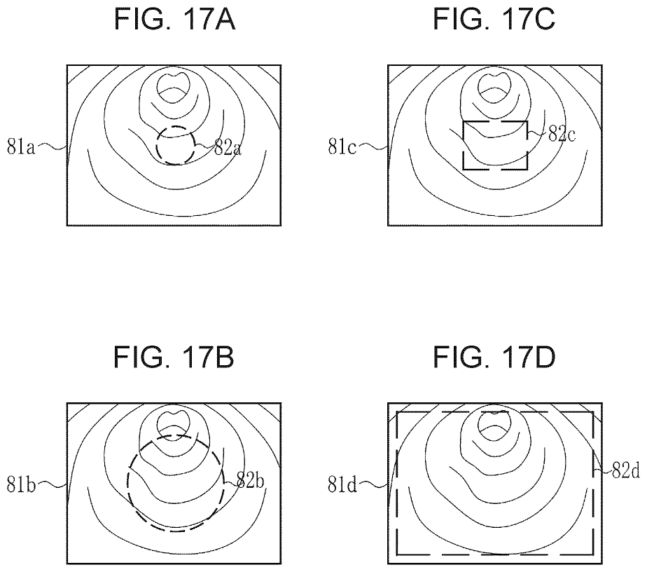

As illustrated in FIGS. 17A to 17D, the region of interest 82 has patterns of a plurality of shapes or sizes. FIGS. 17A to 17D illustrate regions of interest 82a to 82d displayed in the image display region 81a to 81d having the same size and imaging magnification as those in FIG. 10, respectively. FIG. 17A illustrates the region of interest 82a having a circular shape whose area is smaller than that of the region of interest 82. FIG. 17B illustrates the region of interest 82b having a rectangular shape. FIG. 17C illustrates the region of interest 82c having a circular shape whose area is larger than that the region of interest 82. FIG. 17D illustrates the region of interest 82d having a rectangular shape and surrounding substantially the entire image display region 81d. The shape and size of the region of interest 82 may be determined by a user operation, for example, before the correction value calculation operation is performed.

In a case where the area is large, such as in the case of the region of interest 82b or the region of interest 82d, a large number of image signals can be acquired at once to determine a specific pigment concentration. However, inappropriate image signals may be included or it may take time to calculate the specific pigment concentration. In a case where the area is small, such as in the case of the region of interest 82a or the region of interest 82c, by contrast, it is likely to prevent reflected glare of an inappropriate region, and it takes less time to calculate a specific pigment concentration, whereas a smaller number of image signals can be acquired at once. For this reason, it is preferable to selectively use them in accordance with the observation target, the imaging conditions, and so on.

For high-accuracy oxygen saturation observation, even if the area of the region of interest 82 varies, it is preferable to perform adjustment by varying the upper limit number or the lower limit number of specific pigment concentrations to be acquired in the correction value calculation operation in accordance with the area of the region of interest. Preferably, the upper limit number decreases as the area increases, and the lower limit number increases as the area decreases. For example, in a case where the size of the region of interest is large, such as in the case of the region of interest 82d, the upper limit number of specific pigment concentrations to be used for average value calculation is set to three, and in a case where the region of interest 82a having an area less than or equal to a certain value is used, the lower limit number is set to five. Accordingly, it is preferable to read information on the specific pigment from image signals over a certain range of area and calculate an average value of the specific pigment concentrations, regardless of the size of the region of interest. As a more specific example, if the areas of the regions of interest 82a, 82b, 82c, and 82d increase in this order, five to seven specific pigment concentrations are acquired for the region of interest 82a, four to six specific pigment concentrations are acquired for the region of interest 82b, three to four specific pigment concentrations are acquired for the region of interest 82c, and two to three specific pigment concentrations are acquired for the region of interest 82d.

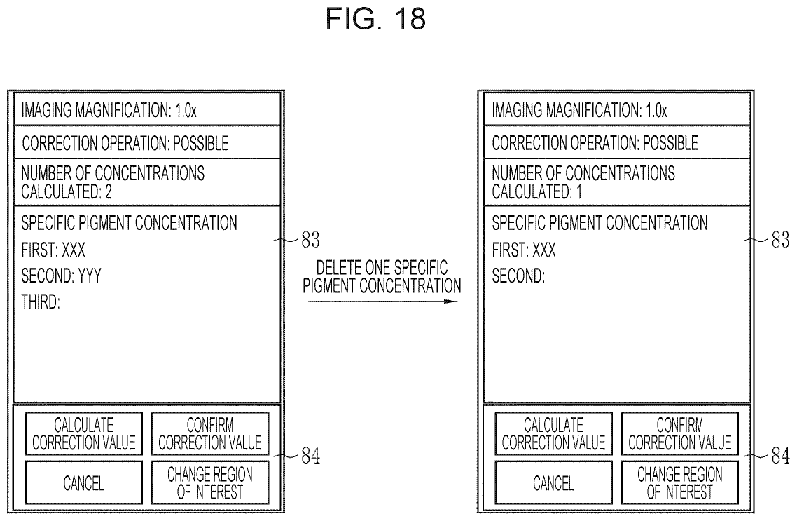

As illustrated in FIG. 18, in the cancellation operation executed in response to a cancellation instruction, the information on the acquired specific pigment concentrations can be canceled. Through the cancellation operation, the information on the immediately previously acquired specific pigment concentration is also deleted from the storage memory 57, and the user can issue a correction value calculation instruction again. The information on the acquired specific pigment concentrations is displayed in the image information display region 83, and any addition or deletion of information is preferably reflected immediately. The information on the specific pigment concentration to be deleted in response to the cancellation operation is not limited to the information on the immediately previously calculated specific pigment concentration, and information on a plurality of specific pigment concentrations stored in the storage memory 57 may be collectively deleted. In this case, preferably, different cancellation instructions are provided in accordance with the length of time over which the tissue-color correction switch 12g is pressed and held. For example, the tissue-color correction switch 12g is pressed for 2 seconds to delete the immediately previously acquired specific pigment concentration, and the tissue-color correction switch 12g is pressed for 4 seconds to collectively delete specific pigment concentrations. In the cancellation operation, furthermore, various operations in the oxygen saturation mode, including a correction value confirmation operation described below, may be canceled.

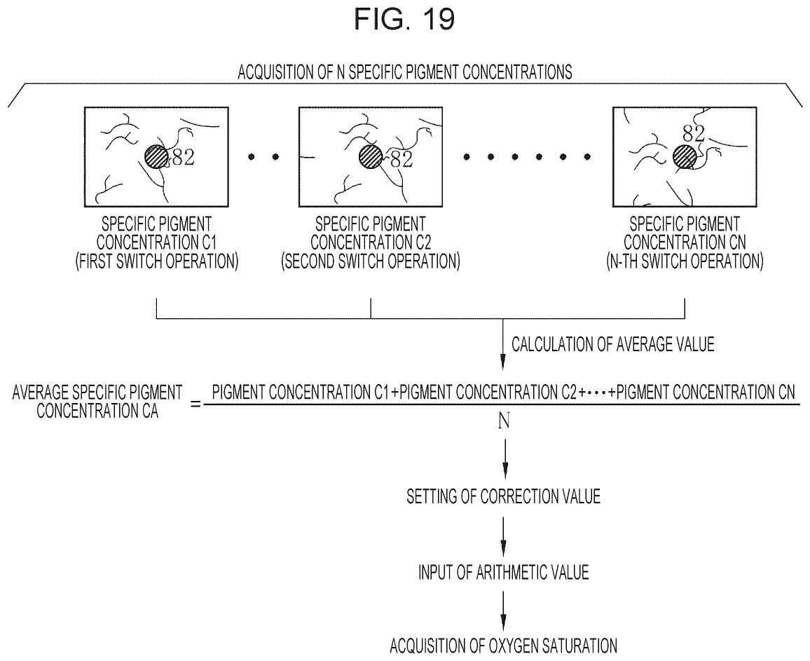

As illustrated in FIG. 19, in the correction value calculation operation for calculating the average specific pigment concentration value CA, N specific pigment concentrations are acquired by a user operation in accordance with the size of the region of interest 82 to calculate an average value. The correction value calculation operation is performed for the first time to acquire a specific pigment concentration C1, the correction value calculation operation is performed for the second time to acquire a specific pigment concentration C2, and the correction value calculation operation is performed for the N-th time to acquire a specific pigment concentration CN. After a plurality of specific pigment concentrations are acquired, in response to a user operation or the acquisition of a certain number of specific pigment concentrations, the correction value calculation operation is terminated, and a correction value confirmation instruction is issued. A correction value confirmation operation is performed, and the total value of the first to N-th specific pigment concentrations is divided by N to calculate the average specific pigment concentration value CA.

A representative value such as the average specific pigment concentration value CA is used to set a correction value for moving the region of the oxygen saturation contour from the reference position. After the correction value is set, the current mode is switched to the oxygen saturation observation mode. In the oxygen saturation observation mode, the acquired arithmetic value is input to obtain the oxygen saturation. Thus, stable oxygen saturation calculation can be performed in real time with a low burden.

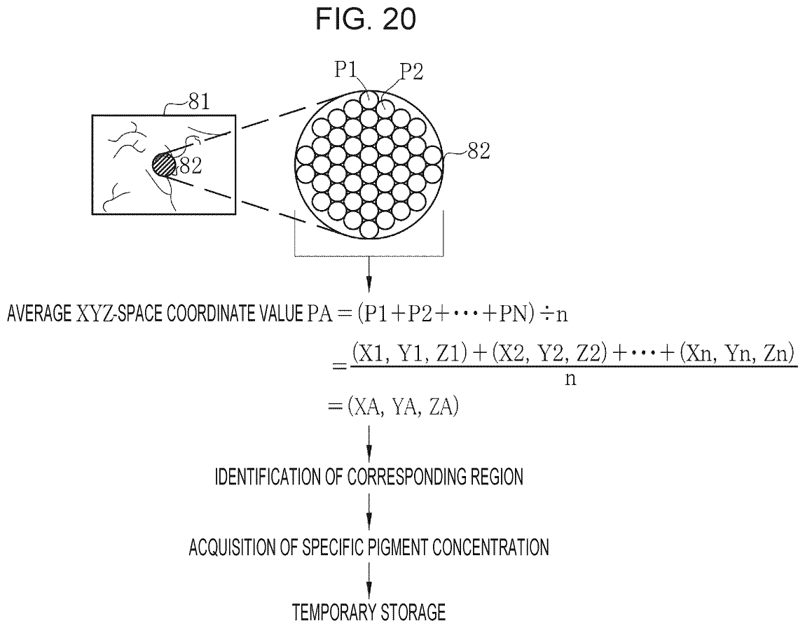

As illustrated in FIG. 20, in the correction value calculation operation, specific pigment concentrations are calculated from image signals obtained in the region of interest 82. For each of the pixels corresponding to the region of interest 82, an X-axis coordinate, a Y-axis coordinate, and a Z-axis coordinate are acquired from the signal ratio R2/G2, the signal ratio B1/G2, and the signal ratio B3/G3, respectively, to calculate coordinate information in the XYZ space. In the correction value calculation operation, an average XYZ-space coordinate value PA for the region of interest 82 is calculated. If the region of interest 82 includes n pixels, a coordinate value P1 is acquired from the first pixel, a coordinate value P2 is acquired from the second pixel, and coordinate value Pn is acquired from the N-th pixel. A corresponding region is calculated from the calculated average XYZ-space coordinate value PA in a way similar to that in FIG. 12, and a correction value for the reference position of the oxygen saturation contour is obtained.

As illustrated in FIG. 14, a region in the XY space, that is, the position of the oxygen saturation contour, is set in accordance with the representative value of the specific pigment concentrations, and the correction value calculation mode is switched to the oxygen saturation observation mode. The switching may be performed automatically after the correction value is confirmed, or may be performed by a user operation.

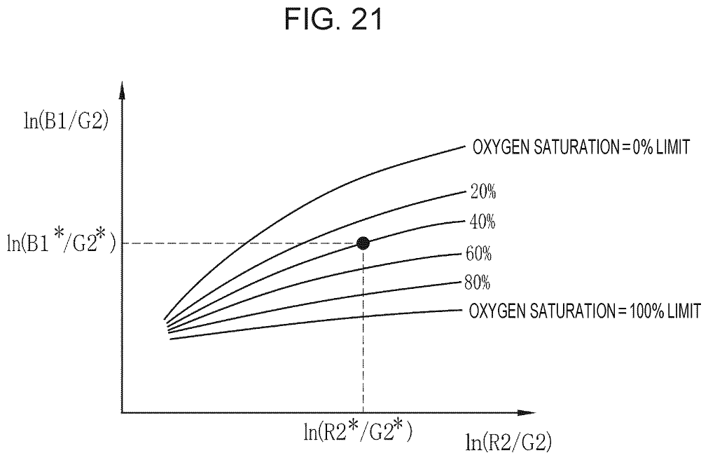

As illustrated in FIG. 21, the oxygen saturation calculation unit 64 refers to the oxygen saturation contours set in accordance with the determined correction value and calculates, for each pixel, an oxygen saturation corresponding to an arithmetic value obtained from the correlation between the signal ratio B1/G2 and the signal ratio R2/G2. For example, the oxygen saturation contour corresponding to an arithmetic value obtained from a signal ratio B1*/G2* and a signal ratio R2*/G2*, which are acquired in the case of the oxygen saturation contour, is “40%”. Accordingly, the oxygen saturation calculation unit 64 calculates the oxygen saturation of the specific pixel as “40%”. While the oxygen saturation contours are displayed at increments of 20%, the oxygen saturation contours may be displayed at increments of 5% or 10%, or enlarged contours centered on the arithmetic value may be used.

The image generation unit 65 uses the oxygen saturation calculated by the oxygen saturation calculation unit 64 to generate an oxygen saturation image in which the oxygen saturation is visualized. Specifically, the image generation unit 65 acquires a B2 image signal, a G2 image signal, and an R2 image signal and applies a gain corresponding to the oxygen saturation to these image signals on a pixel-by-pixel basis. Then, the B2 image signal, the G2 image signal, and the R2 image signal to which the gain is applied are used to generate RGB image data.

For example, for a pixel with an oxygen saturation of 60% or more, the image generation unit 65 multiplies all of the B2 image signal, the G2 image signal, and the R2 image signal obtained in the second frame by the same gain of “1” (corresponding to a normal image). For a pixel with an oxygen saturation of less than 60%, in contrast, the image generation unit 65 multiplies the R2 image signal by a gain less than “1”, and multiplies the B1 image signal and the G2 image signal by a gain greater than “1”. The B1 image signal, the G2 image signal, and the R2 image signal, which are subjected to the gain processing, are used to generate RGB image data that is an oxygen saturation image.

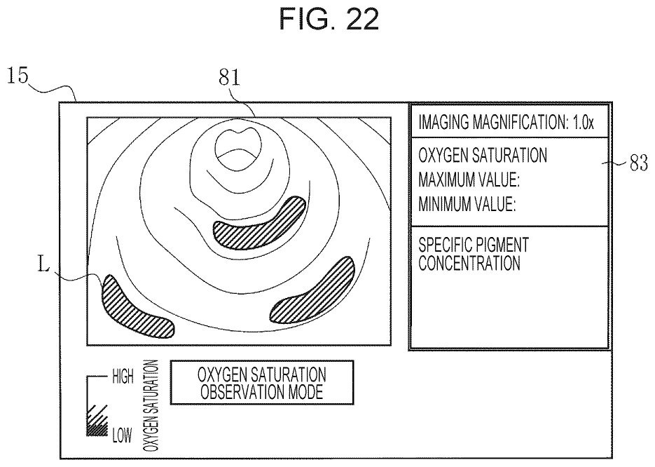

As illustrated in FIG. 22, the oxygen saturation image generated by the image generation unit 65 is displayed in the image display region 81 on the display 15 in such a manner that a region of the oxygen saturation image with an oxygen saturation is represented by a color similar to that of the normal image. In contrast, a region with an oxygen saturation lower than the specific value is represented by a color (pseudo-color) different from that of the normal image and is highlighted as a low-oxygen region L. For example, when the specific value is 60%, a region with an oxygen saturation of 60% to 100% is a high-oxygen region, and a region with an oxygen saturation of 0% to 59% is a low-oxygen region. The specific value may be a fixed value or may be designated by the user in accordance with the content of the examination or the like. The image information display region 83 preferably displays information such as the calculated oxygen saturation. A mode display region provides a display indicating the oxygen saturation observation mode.

The image generation unit 65 according to this embodiment multiplies only a low-oxygen region by a gain for pseudo-color representation. Alternatively, the image generation unit 65 may also multiply a high-oxygen region by a gain corresponding to the oxygen saturation to represent the entire oxygen saturation image by pseudo-color.

The correction value is preferably calculated for each patient or each site. In some cases, for example, the state of pre-processing (the state of the remaining yellow pigment) before endoscopic diagnosis may vary from patient to patient. In such a case, the correlation is adjusted and determined for each patient. In some cases, furthermore, the situation in which the observation target includes a yellow pigment may vary between the observation of the upper digestive tract such as the esophagus or the stomach and the observation of the lower digestive tract such as the large intestine. In such a case, it is preferable to adjust the correlation for each site. In this case, the mode switch 12e is operated to switch from the oxygen saturation observation mode to the correction value calculation mode.

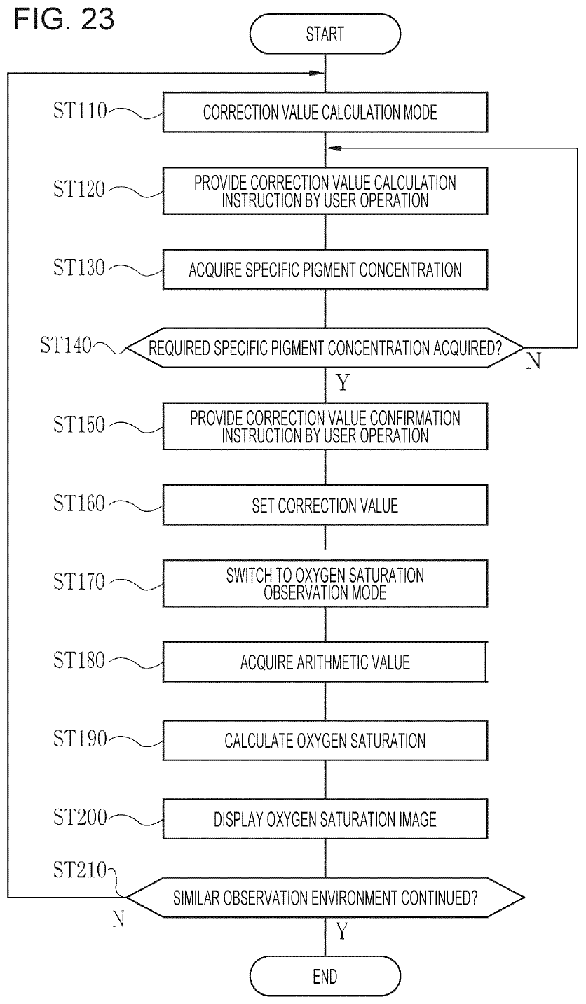

The flow of a series of operations in the oxygen saturation mode will be described with reference to a flowchart in FIG. 23. The user operates the mode switch 12e to set the oxygen saturation mode. Accordingly, the observation target is illuminated with light for three frames having different light emission patterns. Immediately after the switching to the oxygen saturation mode, the correction value calculation mode is set (step ST110). In the correction value calculation mode, the user sets a region of interest in an observation environment for observation including oxygen saturation observation, and presses the tissue-color correction switch 12g once to provide a correction imaging instruction (step ST120). In response to the correction imaging instruction, a correction value calculation operation for acquiring a specific pigment concentration within the range of the region of interest 82 is performed, and information on the acquired specific pigment concentration is temporarily stored (Step ST130). The correction value calculation operation is performed a number of times corresponding to the size of the region of interest 82. If the number of times is insufficient or if an inappropriate specific pigment concentration is acquired (N in step ST140), a correction imaging instruction is provided again (step ST120).

If a plurality of appropriate specific pigment concentrations are acquired (Y in step ST140), the user presses the tissue-color correction switch 12g twice in a row to provide a correction value confirmation instruction (step ST150). In response to the correction value confirmation instruction, a correction value confirmation operation is performed to calculate a representative value such as an average value of the plurality of temporarily stored specific pigment concentrations and set the representative value as a fixed correction value to be used to calculate the oxygen saturation (Step ST160).

After the correction value is set, the correction value calculation mode is switched to the oxygen saturation observation mode by the user operating the mode switch 12e or automatically (step ST170). In the oxygen saturation observation mode, an arithmetic value of the oxygen saturation is acquired from image signals obtained from an image (step ST180). The arithmetic value is corrected using the set correction value to calculate the oxygen saturation (step ST190). The calculated oxygen saturation is visualized as an oxygen saturation image and is displayed on the display 15 (step ST200).

While the observation is continued, if the observation environment does not remain the same and the observation environment changes (N in step ST210), such as if a different site or a different lesion is to be observed, the user operates the mode switch 12e to switch to the correction value calculation mode and set a correction value again (step ST110). If the observation environment remains the same, the observation is continued using the fixed correction value (step ST210). The series of operations described above is repeatedly performed so long as the observation is continued in the oxygen saturation mode.

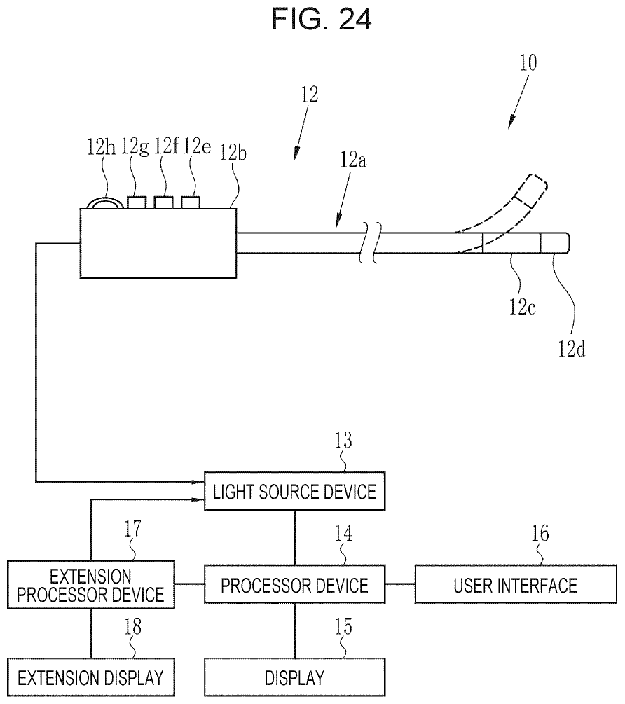

As illustrated in FIG. 24, the endoscope system 10 may be provided with an extension processor device 17, which is different from the processor device 14, and an extension display 18, which is different from the display 15. The extension processor device 17 is electrically connected to the light source device 13, the processor device 14, and the extension display 18. The extension processor device 17 performs processing such as image generation and image display in the oxygen saturation mode. In this case, the extension processor device 17 may implement some of the functions of the processor device 14.

When the extension processor device 17 and the extension display 18 are included, in the oxygen saturation mode, a white-light-equivalent image having fewer short-wavelength components than a white-light image is displayed on the display 15, and the extension display 18 displays an oxygen saturation image that is an image of the oxygen saturation of the observation target that is calculated.

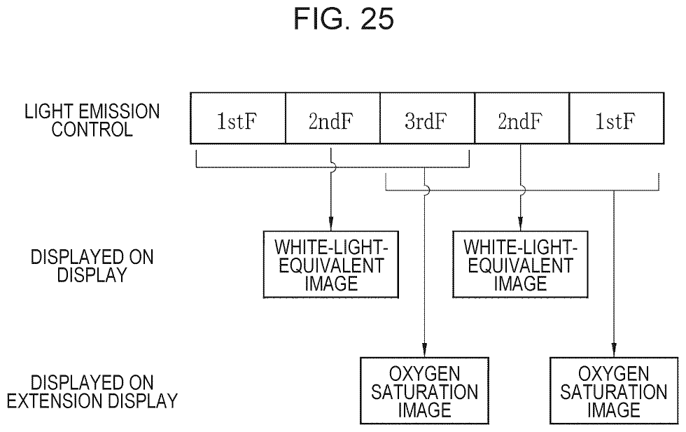

As illustrated in FIG. 25, in the oxygen saturation mode, first illumination light is emitted in the first frame (1stF), second illumination light is emitted in the second frame (2ndF), and third illumination light is emitted in the third frame (3rdF). Thereafter, the second illumination light in the second frame is emitted, and the first illumination light in the first frame is emitted. A white-light-equivalent image obtained in response to emission of the second illumination light in the second frame is displayed on the display 15. Further, an oxygen saturation image obtained in response to emission of the first to third illumination light in the first to third frames is displayed on the extension display 18. When the extension processor device 17 and the extension display 18 are not included, the screen of the display 15 may be divided to perform similar light emission and image display.

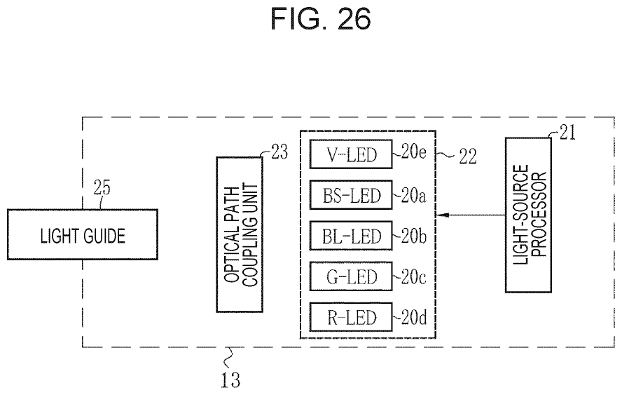

In the normal mode, a white-light-equivalent image formed by the three colors of the short-wavelength blue light BS, the green light G, and the red light R is output. As illustrated in FIG. 26, the light source device 13 may use, in place of the light source unit 20, a light source unit 22 having a V-LED 20e (Violet Light Emitting Diode) that emits violet light V of 410 nm±10 nm to output a white-light image formed by four colors of the violet light V, the short-wavelength blue light BS, the green light G, and the red light R, regardless of the presence or absence of the extension processor device 17 and the extension display 18. In this case, the light-source processor 21 performs light emission control including control of the V-LED 20e that emits the violet light V.

The endoscope 12 used in the endoscope system 10 is of a soft endoscope type for the digestive tract such as the stomach or the large intestine. In the oxygen saturation mode, the endoscope 12 displays an internal-digestive-tract oxygen saturation image that is an image of the state of the oxygen saturation inside the digestive tract. In an endoscope system described below, in the case of a rigid endoscope type for the abdominal cavity such as the serosa, a serosa-side oxygen saturation image that is an image of the state of the oxygen saturation on the serosa side is displayed in the oxygen saturation observation mode. The rigid endoscope type is formed to be rigid and elongated and is inserted into the subject. The serosa-side oxygen saturation image is preferably an image obtained by adjusting the saturation of the white-light-equivalent image. The adjustment of the saturation is preferably performed in the correction value calculation mode regardless of the mucosa or the serosa and the soft endoscope or the rigid endoscope.

The representative value such as the average specific pigment concentration value CA is preferably a weighted average value obtained by weighting the specific pigment concentrations in accordance with the reliability calculated by a reliability calculation unit (not illustrated) described below. In the oxygen saturation mode, the display style of the image display region 81 may be changed in accordance with the reliability. Before performing the correction value calculation operation, it is preferable to select the position of the region of interest 82 on the basis of the reliability visualized in the image display region 81. After the correction value calculation operation is performed, the reliability of the calculated oxygen saturation may be determined in the oxygen saturation observation mode.

Specifically, the image generation unit 65 changes the display style of the image display region 81 so that a difference between a low-reliability region having low reliability and a high-reliability region having high reliability for the calculation of the oxygen saturation is emphasized. The reliability indicates the calculation accuracy of the oxygen saturation for each pixel, with higher reliability indicating higher calculation accuracy of the oxygen saturation. The low-reliability region is a region having reliability less than a reliability threshold value. The high-reliability region is a region having reliability greater than or equal to the reliability threshold value. In an image for correction, emphasizing the difference between the low-reliability region and the high-reliability region enables the specific region to include the high-reliability region while avoiding the low-reliability region.

The reliability is calculated by a reliability calculation unit included in the oxygen saturation image processing unit 55. Specifically, the reliability calculation unit calculates at least one reliability that affects the calculation of the oxygen saturation on the basis of the B1 image signal, the G1 image signal, and the R1 image signal acquired in the first frame or the B2 image signal, the G2 image signal, and the R2 image signal acquired in the second frame. The reliability is represented by, for example, a decimal number between 0 and 1. In a case where the reliability calculation unit calculates a plurality of types of reliabilities, the reliability of each pixel is preferably the minimum reliability among the plurality of types of reliabilities.

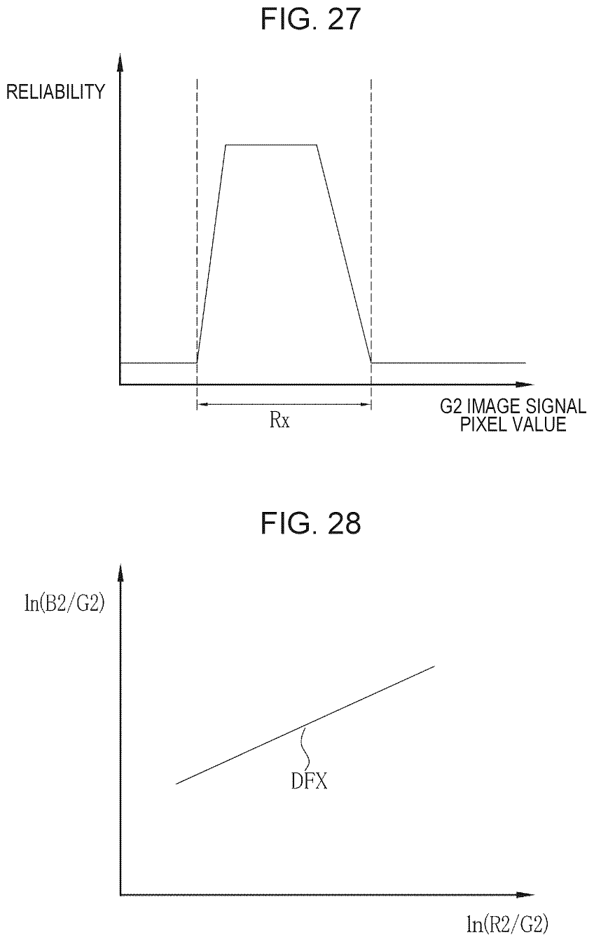

As illustrated in FIG. 27, for example, for a brightness value that affects the calculation accuracy of the oxygen saturation, the reliability for a brightness value of a G2 image signal outside a certain range Rx is lower than the reliability for a brightness value of a G2 image signal within the certain range Rx. The case of being outside the certain range Rx is a case of a high brightness value such as halation, or is a case of a very low brightness value such as in a dark portion. As described above, the calculation accuracy of the oxygen saturation is low for a brightness value outside the certain range Rx, and the reliability is also low accordingly.

The calculation accuracy of the oxygen saturation is affected by a disturbance, examples of which includes at least bleeding, fat, a residue, mucus, or a residual liquid, and such a disturbance may also cause a variation in reliability. For bleeding, which is one of the disturbances described above, as illustrated in FIG. 28, the reliability is determined in accordance with a distance from a definition line DFX in a two-dimensional plane defined by a vertical axis ln (B2/G2) and a horizontal axis ln (B2/G2). As the distance from the definition line DFX to coordinates plotted on the two-dimensional plane on the basis of the B1 image signal, the G1 image signal, and the R1 image signal increases, the reliability decreases. For example, the closer the coordinates plotted on the two-dimensional plane are to the lower right, the lower the reliability.

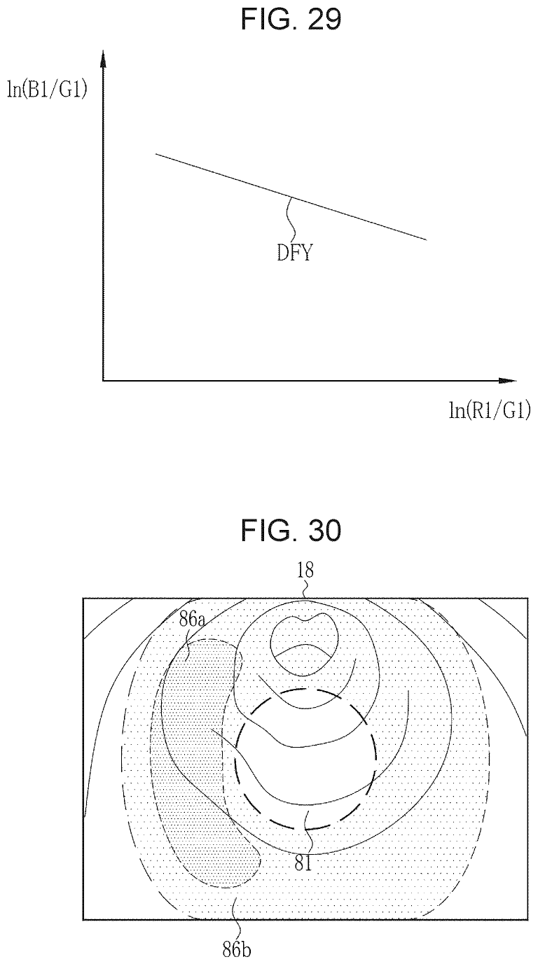

For fat, a residue, a residual liquid, or mucus, which is included in the disturbances described above, as illustrated in FIG. 29, the reliability is determined in accordance with a distance from a definition line DFY in a two-dimensional plane defined by a vertical axis ln (B1/G1) and a horizontal axis ln (B1/G1). As the distance from the definition line DFY to coordinates plotted on the two-dimensional plane on the basis of the B2 image signal, the G2 image signal, and the R2 image signal increases, the reliability decreases. For example, the closer the coordinates plotted on the two-dimensional plane are to the lower left, the lower the reliability.

In a method by which the image generation unit 65 emphasizes a difference between a low-reliability region and a high-reliability region, as illustrated in FIG. 30, the image generation unit 65 sets the saturation of a low-reliability region 86a to be higher than the saturation of a high-reliability region 86b. This allows the user to easily select the high-reliability region 86b as the region of interest 82 while avoiding the low-reliability region 86a. Further, the image generation unit 65 reduces the luminance of a dark portion in the low-reliability region 86a. This allows the user to easily avoid the dark portion when selecting the position of the region of interest 82. The dark portion is a dark region having a brightness value less than or equal to a certain value. The low-reliability region 86a and the high-reliability region 86b may have opposite colors.

The image generation unit 65 preferably changes the display style of the specific region in accordance with the reliability in the specific region. In the correction value calculation mode, before the correction value calculation operation is performed, it is determined whether it is possible to appropriately perform correction processing on the basis of the reliability in the region of interest 82. If the number of effective pixels having reliability greater than or equal to the reliability threshold value among the pixels in the specific region is greater than or equal to a certain value, it is determined that it is possible to appropriately perform the correction processing. On the other hand, if the number of effective pixels among the pixels in the specific region is less than the certain value, it is determined that it is not possible to appropriately perform the correction processing. The determination is preferably performed each time an image is acquired and the reliability is calculated until a correction operation is performed. The period in which the determination is performed may be changed as appropriate.

In the correction value calculation mode, after the correction operation has been performed, it is determined whether it is possible to appropriately perform correction processing on the basis of the reliability in the specific region at the timing when the correction operation was performed. It is also preferable to provide a notification related to the determination result.

On the other hand, if it is determined that it is not possible to appropriately perform the correction processing, a notification is provided indicating that another correction operation is required since it is not possible to appropriately perform the correction processing. For example, a message such as “Another correction operation is required” is displayed. In this case, in addition to or instead of the message, a notification of operational guidance for performing appropriate table correction processing is preferably provided. Examples of the notification include a notification of operational guidance such as “Please avoid the dark portion” and a notification of operational guidance such as “Please avoid bleeding, a residual liquid, fat, and so on”.

### Second Embodiment

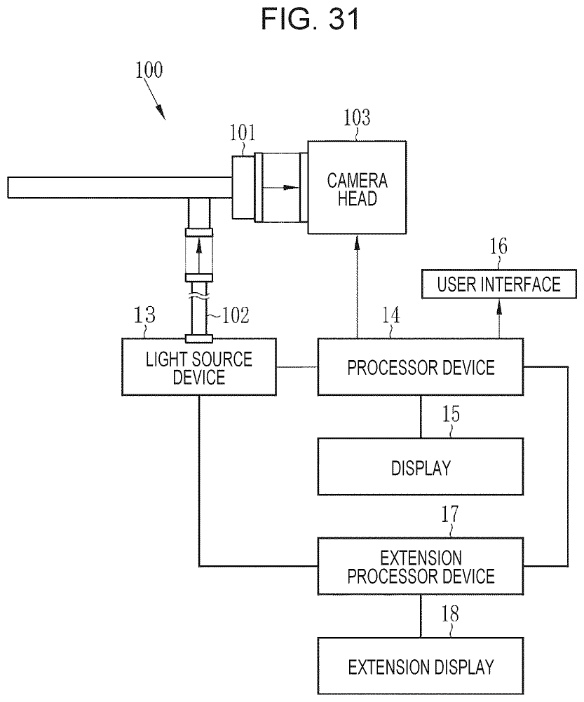

In the first embodiment, the endoscope 12, which is a soft endoscope for digestive-tract endoscopy, is used. Alternatively, an endoscope serving as a rigid endoscope for laparoscopic endoscopy may be used. In the use of an endoscope that is a rigid endoscope, an endoscope system 100 illustrated in FIG. 31 is used. The endoscope system 100 includes an endoscope 101, a light guide 102, a light source device 13, a processor device 14, a display 15, a user interface 16, an extension processor device 17, and an extension display 18. In the following, portions of the endoscope system 100 common to those of the first embodiment will not be described, and only different portions will be described.

The endoscope 101, which is used for laparoscopic surgery or the like, is formed to be rigid and elongated and is inserted into a subject. A camera head 103 is attached to the endoscope 101 and is configured to perform imaging of the observation target on the basis of reflected light guided from the endoscope 101. An image signal obtained by the camera head 103 through imaging is transmitted to the processor device 14.

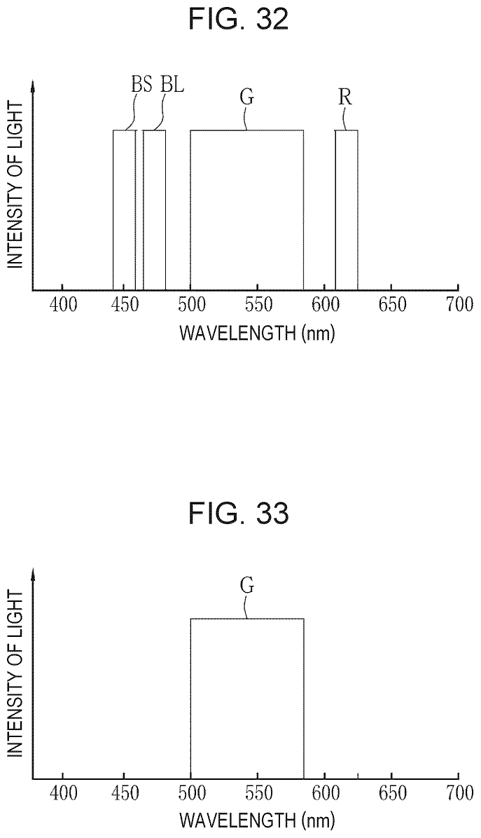

The light emission control in the oxygen saturation mode according to this embodiment is to perform imaging (white frame W) with radiation of four-color mixed light that is white light generated by the LEDs 20a to 20d, as illustrated in FIG. 32, and imaging (green frame Gr) with only the LED 20c turned on to emit green light G, as illustrated in FIG. 33, and the light is emitted from the distal end of the endoscope 101 toward the photographic subject via the light guide 102. The white light and the green light are emitted in a switching manner in accordance with a specific light emission pattern. Thereafter, the photographic subject is irradiated with the illumination light, and return light from the photographic subject is guided to the camera head 103 via an optical system (optical system for forming an image of the photographic subject) incorporated in the endoscope 101. In the normal mode, imaging is performed using four-color mixed light or imaging is performed using normal light (white light) obtained by adding the violet light V to the four-color mixed light.

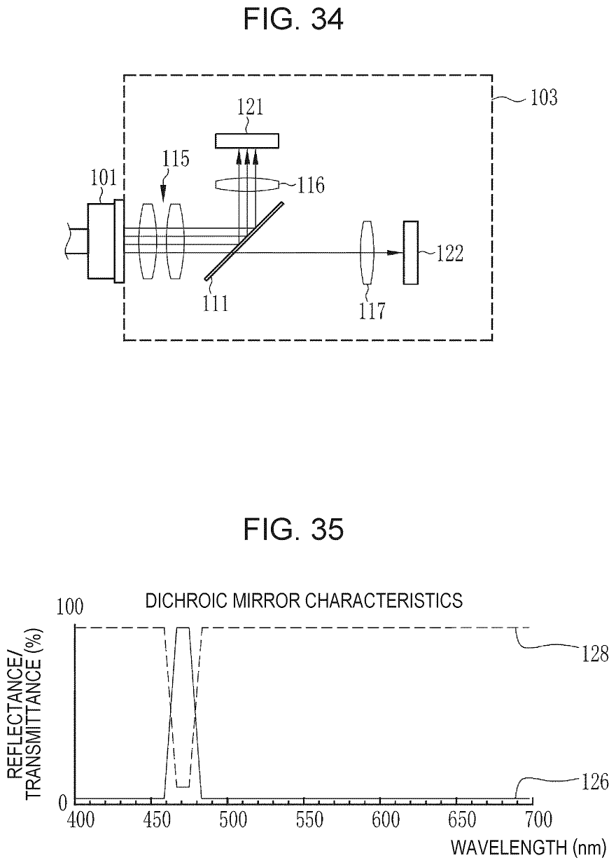

As illustrated in FIG. 34, the camera head 103 includes a dichroic mirror (spectral element) 111, image-forming optical systems 115, 116, and 117, and CMOS (Complementary Metal Oxide Semiconductor) sensors, namely, a color imaging sensor 121 (normal imaging element) and a monochrome imaging sensor 122 (specific imaging element). Light entering the camera head 103 includes light reflected by the dichroic mirror 111 and incident on the color imaging sensor 121, and light transmitted through the dichroic mirror 111 and incident on the monochrome imaging sensor 122.

In FIG. 35, as indicated by a solid line 126 (transmission characteristic line), the dichroic mirror 111 has a property of transmitting return light of the long-wavelength blue light BL (light having a center wavelengths of about 470 nm) with which the photographic subject is irradiated. In contrast, as indicated by a broken line 128 (reflection characteristic line), the dichroic mirror 111 has a property of reflecting mixed light including, specifically, return light of the short-wavelength blue light BS (light with a center wavelength of about 450 nm), return light of the green light G (light with a center wavelength of about 540 nm), and return light of the red light R (light with a center wavelength of about 640 nm).

As indicated by the solid line 126 (transmission characteristic line), a spectral element including the dichroic mirror 111 can typically reduce the transmittance of light in a desired wavelength range to substantially 0%, and more specifically, to about 0.1%. In contrast, as indicated by the broken line 128, it is difficult to reduce the reflectance of light in a desired wavelength range to substantially 0%, and the spectral element has a property of reflecting approximately 2% of light in a wavelength range that is not intended to be reflected.

As described above, the light reflected by the dichroic mirror 111 also includes light in a wavelength range that is not intended to be reflected. Thus, in a configuration that allows the dichroic mirror 111 to reflect return light of the long-wavelength blue light BL, the return light of the long-wavelength blue light BL is mixed with return light of the normal light. In contrast, the present invention provides a configuration that allows the dichroic mirror 111 to transmit the return light of the long-wavelength blue light BL. This configuration makes it possible to prevent mixing of return light of light other than the long-wavelength blue light BL (as compared with the configuration that allows the dichroic mirror 111 to reflect return light of the long-wavelength blue light BL, mixing of return light of light other than the long-wavelength blue light BL can be reduced to about 1/20).

Of the return light of the four-color mixed light, the light (mixed light) reflected by the dichroic mirror 111 is incident on the color imaging sensor 121, and in this process, an image is formed on an imaging surface of the color imaging sensor 121 by the image-forming optical systems 115 and 116. The return light of the long-wavelength blue light BL, which is light transmitted through the dichroic mirror 111, is imaged by the image-forming optical systems 115 and 117 in the process of being incident on the monochrome imaging sensor 122, and an image is formed on an imaging surface of the monochrome imaging sensor 122.

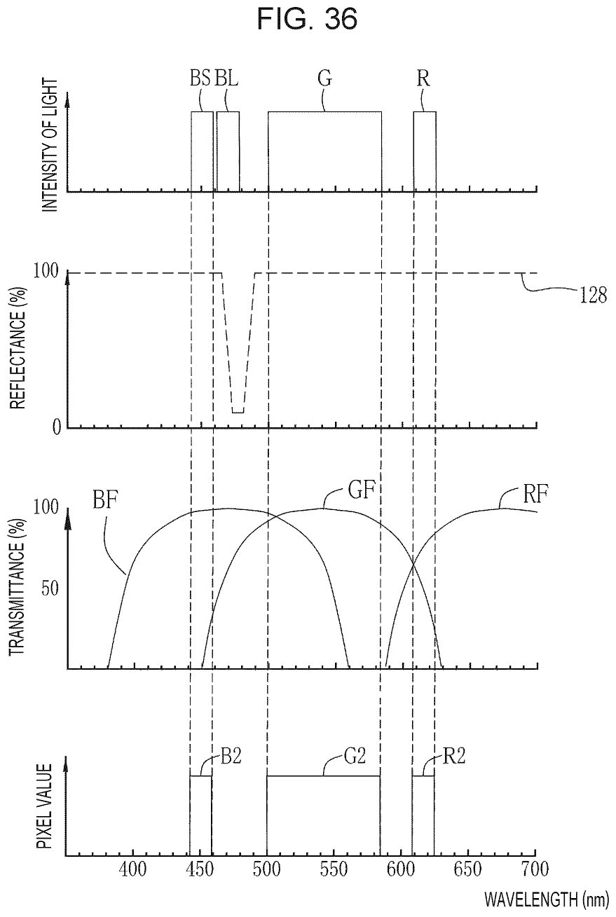

The imaging (white frame W) with radiation of four-color mixed light, which is white light, will be described. In light reception by the color imaging sensor 121, the light source unit 20 emits light from the four color LEDs (simultaneously emits blue light and white light), and the return light thereof enters the camera head 103. As illustrated in FIG. 36, of the entering light, the return light of the mixed light other than the return light of the long-wavelength blue light BL is reflected by the dichroic mirror 111. The reflected light is incident on each of the pixels arrayed across the color imaging sensor 121. In the color imaging sensor 121, the B pixels output a B2 image signal having a pixel value corresponding to light transmitted through the B color filter BF out of the short-wavelength blue light BS. The G pixels output a G2 image signal having a pixel value corresponding to light transmitted through the G color filter GF out of the green light G. The R pixels output an R2 image signal having a pixel value corresponding to light transmitted through the R color filter RF out of the red light R.

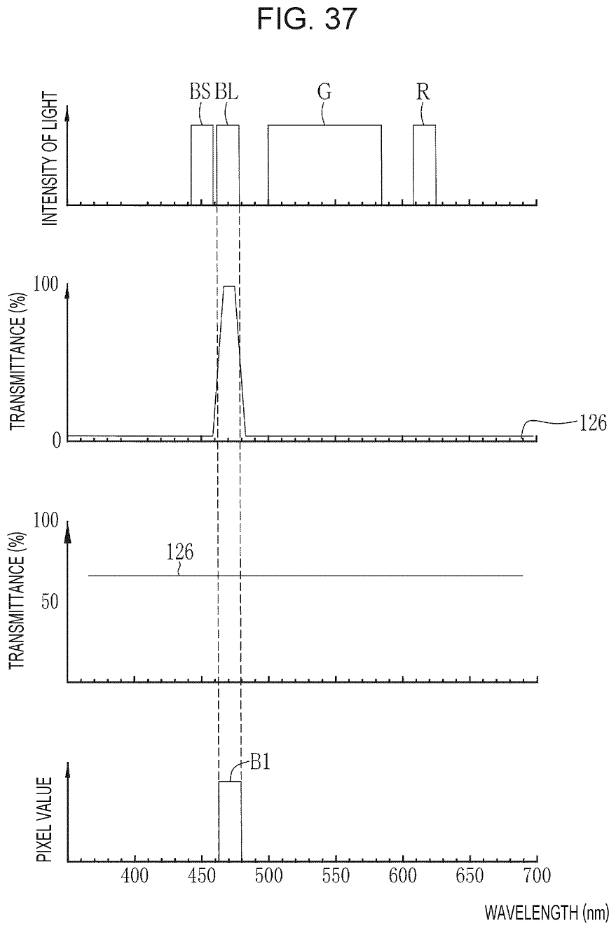

The reception of light by the monochrome imaging sensor 122 when light is emitted from the four color LEDs will be described. The light source unit 20 emits light from the four color LEDs (simultaneously emits blue light and white light), and the return light thereof enters the camera head 103. As illustrated in FIG. 37, the return light of the long-wavelength blue light BL out of the entering light is transmitted through the dichroic mirror 111. The transmitted light is incident on the monochrome pixels arrayed across the monochrome imaging sensor 122. The monochrome imaging sensor 122 outputs a B1 image signal having a pixel value corresponding to the incident long-wavelength blue light BL.

In this embodiment, the color imaging sensor 121 and the monochrome imaging sensor 122 perform imaging to simultaneously obtain a monochrome image (oxygen saturation image) from the B1 image signal (monochrome image signal) and a white-light-equivalent image (observation image) from the R2 image signal, the G2 image signal, and the B2 image signal. Since the observation image and the oxygen saturation image are obtained simultaneously (obtained from images captured at the same timing), no need exists to perform processing such as registration of the two images when, for example, the two images are to be displayed in a superimposed manner later.

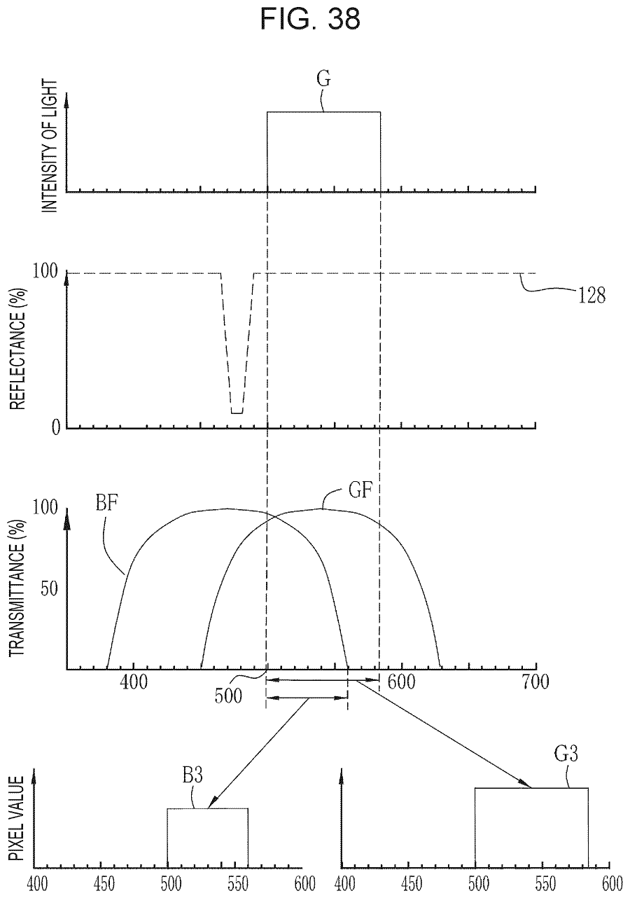

In contrast, as illustrated in FIG. 38, in imaging with radiation of the green light G (green frame Gr), upon emission of only the green light G, the green light G incident on the camera head 103 is reflected by the dichroic mirror 111 and is incident on the color imaging sensor 121. In the color imaging sensor 121, the B pixels output a B3 image signal having a pixel value corresponding to light transmitted through the B color filter BF out of the green light G. The G pixels output a G3 image signal having a pixel value corresponding to light transmitted through the G color filter GF out of the green light G. In the green frame, the image signals output from the monochrome imaging sensor 122 and the image signals output from the R pixels of the color imaging sensor 121 are not used in the subsequent processing steps.

In imaging, the processor device 14 drives the color imaging sensor 121 and the monochrome imaging sensor 122 to continuously perform imaging in a preset imaging cycle (frame rate). In imaging, furthermore, the processor device 14 controls the shutter speed of an electronic shutter, that is, the exposure period, of each of the color imaging sensor 121 and the monochrome imaging sensor 122 independently for each of the imaging sensors 121 and 122. As a result, the luminance of an image obtained by the color imaging sensor 121 and/or the monochrome imaging sensor 122 is controlled (adjusted).

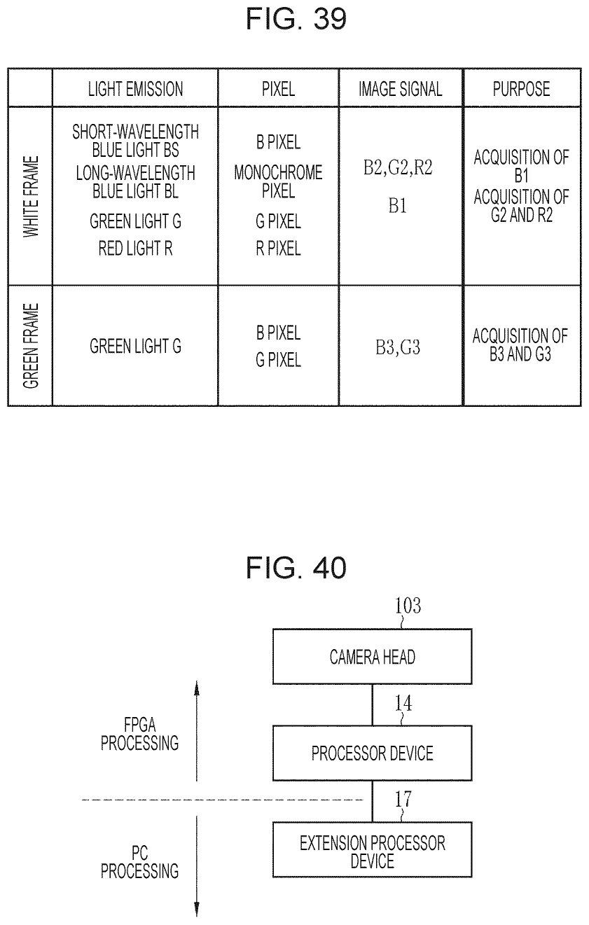

As illustrated in FIG. 39, as described above, in a white frame, a B2 image signal, a G2 image signal, and an R2 image signal are output from the color imaging sensor 121, and a B1 image signal is output from the monochrome imaging sensor 122. The B1, B2, G2, and R2 image signals are used in the subsequent processing steps. In a green frame, by contrast, a B3 image signal and a G3 image signal are output from the color imaging sensor 121 and are used in the subsequent processing steps.

As illustrated in FIG. 40, the image signals output from the camera head 103 are sent to the processor device 14, and data on which various types of processing are performed by the processor device 14 is sent to the extension processor device 17. When the endoscope 101 is used, the processing load on the processor device 14 is taken into account, and the processes are performed in the oxygen saturation mode such that the processor device 14 performs low-load processing and then the extension processor device 17 performs high-load processing. Of the processes to be performed in the oxygen saturation mode, the processing to be performed by the processor device 14 is mainly performed by an FPGA (Field-Programmable Gate Array) and is thus referred to as FPGA processing. On the other hand, the processing to be performed by the extension processor device 17 is referred to as PC processing since the extension processor device 17 is implemented as a PC (Personal Computer).

When the endoscope 101 is provided with an FPGA (not illustrated), the FPGA of the endoscope 101 may perform the FPGA processing. While the following describes the FPGA processing and the PC processing in the correction mode, the processes are preferably divided into the FPGA processing and the PC processing also in the oxygen saturation mode to share the processing load.

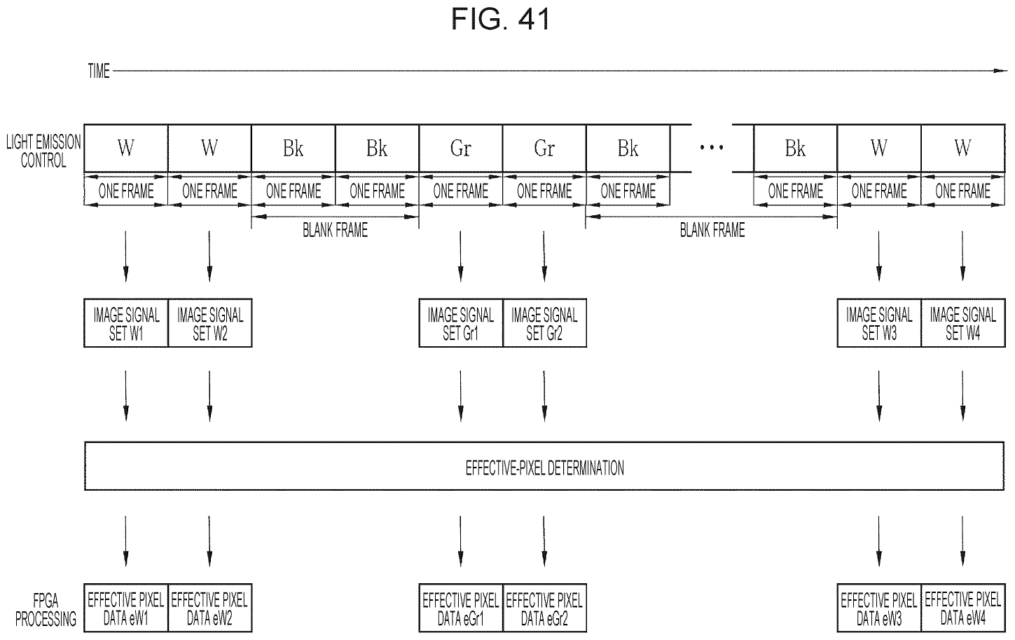

In a case where the endoscope 101 is used and light emission control is performed for a white frame W and a green frame Gr in accordance with a specific light emission pattern, as illustrated in FIG. 41, the specific light emission pattern is such that light is emitted in two white frames W and then two blank frames Bk are used in which no light is emitted from the light source device 13. Thereafter, light is emitted in two green frames Gr, and then two or more several (e.g., seven) blank frames Bk are used. Thereafter, light is emitted again in two white frames W. The specific light emission pattern described above is repeatedly performed. As in the specific light emission pattern described above, light is emitted in the white frame W and the green frame Gr at least in the correction value calculation mode. In the oxygen saturation observation mode, light may be emitted in only the white frame W, but no light is emitted in the green frame Gr.

In the following, of the first two white frames, the first white frame is referred to as a white frame W1, and the subsequent white frame is referred to as a white frame W2 to distinguish the light emission frames in which light is emitted in accordance with a specific light emission pattern. Of the two green frames, the first green frame is referred to as a green frame Gr1, and the subsequent green frame is referred to as a green frame Gr2. Of the last two white frames, the first white frame is referred to as a white frame W3, and the subsequent white frame is referred to as a white frame W4.

The image signals for the correction value calculation mode (the B1 image signal, the B2 image signal, the G2 image signal, the R2 image signal, the B3 image signal, and the G3 image signal) obtained in the white frame W1 are referred to as an image signal set W1. Likewise, the image signals for the correction mode obtained in the white frame W2 are referred to as an image signal set W2. The image signals for the correction mode obtained in the green frame Gr1 are referred to as an image signal set Gr1. The image signals for the correction mode obtained in the green frame Gr2 are referred to as an image signal set Gr2. The image signals for the correction mode obtained in the white frame W3 are referred to as an image signal set W3. The image signals for the correction mode obtained in the white frame W4 are referred to as an image signal set W4. The image signals for the oxygen saturation mode are image signals included in a white frame (the B1 image signal, the B2 image signal, the G2 image signal, and the R2 image signal).

In the FPGA processing, the pixels of all the image signals included in the image signal sets W1, W2, Gr1, Gr2, W3, and W4 are subjected to effective-pixel determination to determine whether the processing can be accurately performed in the oxygen saturation observation mode or the correction value calculation mode. The number of blank frames Bk between the white frame W and the green frame Gr is desirably about two because it is only required to eliminate the light other than the green light G, whereas the number of blank frames Bk between the green frame Gr and the white frame W is two or more because it is necessary to take time to stabilize the light emission state because of the start of turning on the light other than the green light G.

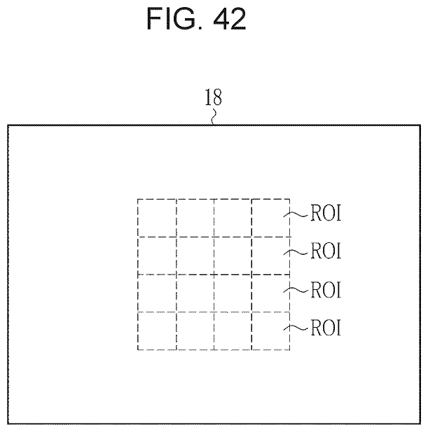

As illustrated in FIG. 42, the effective-pixel determination is performed on the basis of pixel values in 16 center regions ROI provided in a center portion of an image. Specifically, for each of the pixels in the center regions ROI, if the pixel value falls within a range between an upper limit threshold value and a lower limit threshold value, the pixel is determined to be an effective pixel. The effective-pixel determination is performed on the pixels of all the image signals included in the image signal sets. The upper limit threshold value or the lower limit threshold value is set in advance in accordance with the sensitivity of the B pixels, the G pixels, and the R pixels of the color imaging sensor 121 or the sensitivity of the monochrome imaging sensor 122.

On the basis of the effective-pixel determination described above, the number of effective pixels, the total pixel value of the effective pixels, and the sum of squares of the pixel values of the effective pixels are calculated for each of the center regions ROI. The number of effective pixels, the total pixel value of the effective pixels, and the sum of squares of the pixel values of the effective pixels for each of the center regions ROI are output to the extension processor device 17 as each of pieces of effective pixel data eW1, eW2, eGr1, eGr2, eW3, and eW4.

The FPGA processing is arithmetic processing using image signals of the same frame, such as effective-pixel determination, and has a lighter processing load than arithmetic processing using inter-frame image signals of different light emission frames, such as PC processing described below. The pieces of effective pixel data eW1, eW2, eGr1, eGr2, eW3, and eW4 correspond to pieces of data obtained by performing effective-pixel determination on all the image signals included in the image signal sets W1, W2, Gr1, Gr2, W3, and W4, respectively.

In the PC processing, intra-frame PC processing and inter-frame PC processing are performed on image signals of the same frame and image signals of different frames, respectively, among the pieces of effective pixel data eW1, eW2, eGr1, eGr2, eW3, and eW4. In the intra-frame PC processing, the average value of pixel values, the standard deviation value of the pixel values, and the effective pixel rate in the center regions ROI are calculated for all the image signals included in each piece of effective pixel data. The average value of the pixel values and the like in the center regions ROI, which are obtained by the intra-frame PC processing, are used in an arithmetic operation for obtaining a specific result in the oxygen saturation observation mode or the correction value calculation mode.

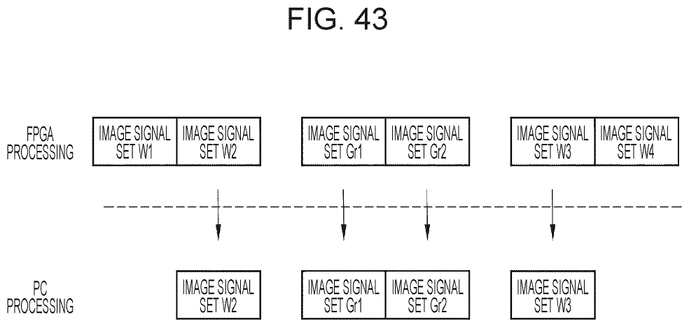

In the inter-frame PC processing, as illustrated in FIG. 43, among the pieces of effective pixel data eW1, eW2, eGr1, eGr2, eW3, and eW4 obtained in the FPGA processing, effective pixel data having a short time interval between the white frame and the green frame is used, and the other effective pixel data is not used in the inter-frame PC processing. Specifically, a pair of the effective pixel data eW2 and the effective pixel data eGr1 and a pair of the effective pixel data eGr2 and the effective pixel data eW3 are used in the inter-frame PC processing. The other pieces of effective pixel data eW1 and eW4 are not used in the inter-frame PC processing. The use of a pair of image signals having a short time interval provides accurate inter-frame PC processing without misalignment of pixels.

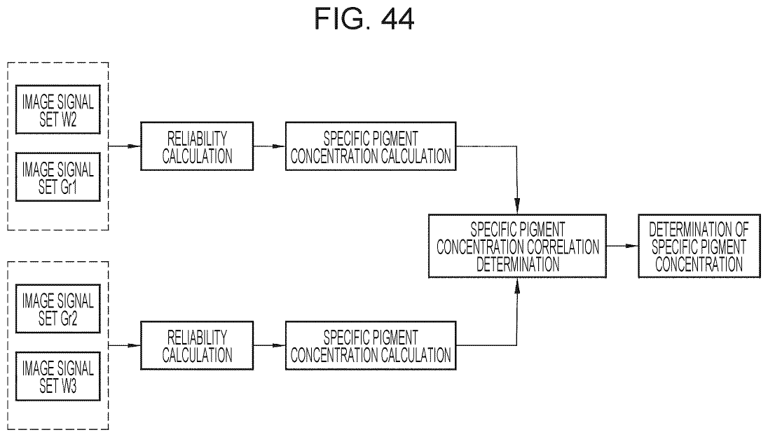

As illustrated in FIG. 44, the inter-frame PC processing using the pair of the effective pixel data eW2 and the effective pixel data eGr1 involves reliability calculation and specific pigment concentration calculation, and the inter-frame PC processing using the pair of the effective pixel data eGr2 and the effective pixel data eW3 also involves reliability calculation and specific pigment concentration calculation. Then, specific pigment concentration correlation determination is performed on the basis of the calculated specific pigment concentrations.

In the calculation of the reliability, the reliability is calculated for each of the 16 center regions ROI. The method for calculating the reliability is similar to the calculation method performed by the reliability calculation unit according to the first embodiment. For example, the reliability for a brightness value of a G2 image signal outside the certain range Rx is preferably set to be lower than the reliability for a brightness value of a G2 image signal within the certain range Rx (see FIG. 27). In the case of the pair of the effective pixel data eW2 and the effective pixel data eGr1, a total of 32 reliabilities are calculated by reliability calculation of a G2 image signal included in each piece of effective pixel data for each of the center regions ROI. Likewise, in the pair of the effective pixel data eGr2 and the effective pixel data eW3, a total of 32 reliabilities are calculated. When the reliability is calculated, for example, if a center region ROI having low reliability is present or if the average reliability value of the center regions ROI is less than a predetermined value, error determination is performed for the reliability. The result of the error determination for the reliability is displayed on the extension display 18 or the like to provide a notification to the user.

In the specific pigment concentration calculation, a specific pigment concentration is calculated for each of the 16 center regions ROI. The method for calculating the specific pigment concentration is similar to the calculation method performed by the specific pigment concentration acquisition unit 61 described above. For example, a specific pigment concentration calculation table 62a is referred to by using the B1 image signal, the G2 image signal, the R2 image signal, the B3 image signal, and the G3 image signal included in the effective pixel data eW2 and the effective pixel data eGr1, and a specific pigment concentration corresponding to the signal ratios ln (B1/G2), In (G2/R2), and ln (B3/G3) is calculated. As a result, a total of 16 specific pigment concentrations PG1 are calculated for the respective center regions ROI. Also in the case of the pair of the effective pixel data eGr2 and the effective pixel data eW3, a total of 16 specific pigment concentrations PG2 are calculated for the respective center regions ROI in a similar manner.

When the specific pigment concentrations PG1 and the specific pigment concentrations PG2 are calculated, correlation values between the specific pigment concentrations PG1 and the specific pigment concentrations PG2 are calculated for the respective center regions ROI. The correlation values are preferably calculated for the respective center regions ROI at the same position. If a certain number or more of center regions ROI having correlation values lower than a predetermined value are present, it is determined that a motion has occurred between the frames, and error determination for the motion is performed. The result of the error determination for the motion is notified to the user by, for example, being displayed on the extension display 18.

If no error is present in the error determination for the motion, one specific pigment concentration is calculated from among the total of 32 specific pigment concentrations PG1 and specific pigment concentrations PG2 by using a specific estimation method (e.g., a robust estimation method). The calculated specific pigment concentration is used in the correction processing for the correction mode. The correction processing for the correction mode is similar to that described above, such as table correction processing.

### Third Embodiment

When the endoscope system 100 (see FIG. 31), which is a rigid endoscope for laparoscopic endoscopy, is used, the endoscope system 100 may include, in place of the camera head 103 that performs imaging with two imaging sensors according to the second embodiment, a camera head 203 that performs imaging of the observation target by an imaging method using four monochrome imaging sensors. In the following, portions of the endoscope system 100 common to those of the first and second embodiments will not be described, and only different portions will be described.

In the normal mode, the light source device 13 (see FIG. 26) including the light source unit 22 having the V-LED 20e supplies white light including the violet light V, the short-wavelength blue light BS, the green light G, and the red light R to the endoscope 101. In the oxygen saturation mode, as illustrated in FIG. 32, the light source device 13 emits mixed light including the long-wavelength blue light BL, the short-wavelength blue light BS, the green light G, and the red light R and supplies the mixed light to the endoscope 101.

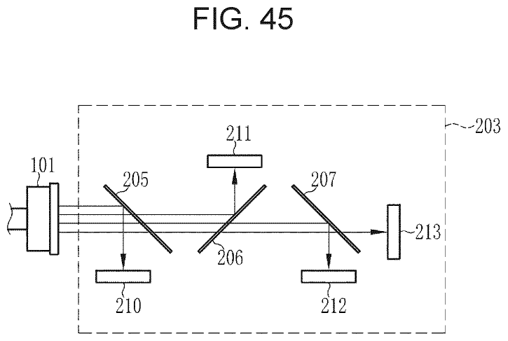

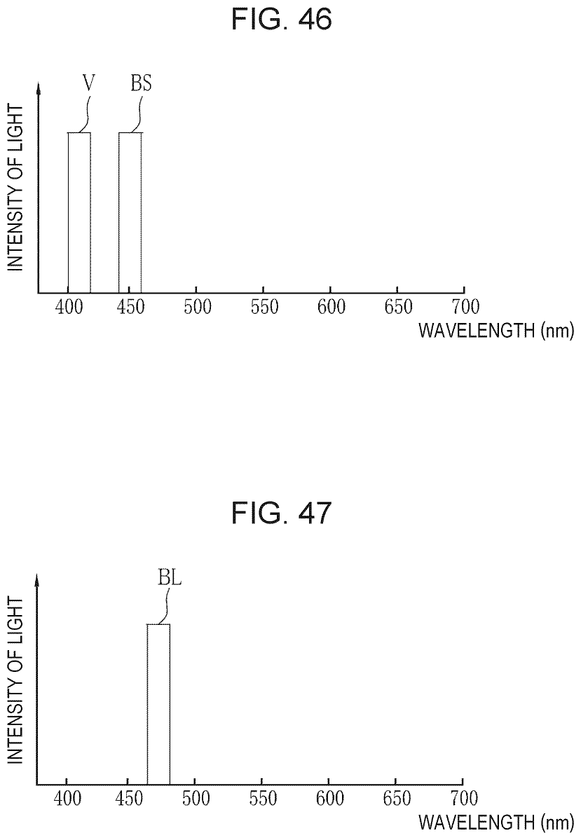

As illustrated in FIG. 45, the camera head 203 includes dichroic mirrors 205, 206, and 207, and monochrome imaging sensors 210, 211, 212, and 213. The dichroic mirror 205 reflects, of the reflected light of the mixed light from the endoscope 101, the violet light V and the short-wavelength blue light BS and transmits the long-wavelength blue light BL, the green light G, and the red light R. As illustrated in FIG. 46, the violet light V or the short-wavelength blue light BS reflected by the dichroic mirror 205 is incident on the imaging sensor 210. The imaging sensor 210 outputs a Bc image signal in response to the incidence of the violet light V and the short-wavelength blue light BS in the normal mode, and outputs a B2 image signal in response to the incidence of the short-wavelength blue light BS in the oxygen saturation mode.

The dichroic mirror 206 reflects, of the light transmitted through the dichroic mirror 205, the long-wavelength blue light BL and transmits the green light G and the red light R. As illustrated in FIG. 47, the long-wavelength blue light BL reflected by the dichroic mirror 206 is incident on the imaging sensor 211. The imaging sensor 211 stops outputting an image signal in the normal mode, and outputs a B1 image signal in response to the incidence of the long-wavelength blue light BL in the oxygen saturation mode.

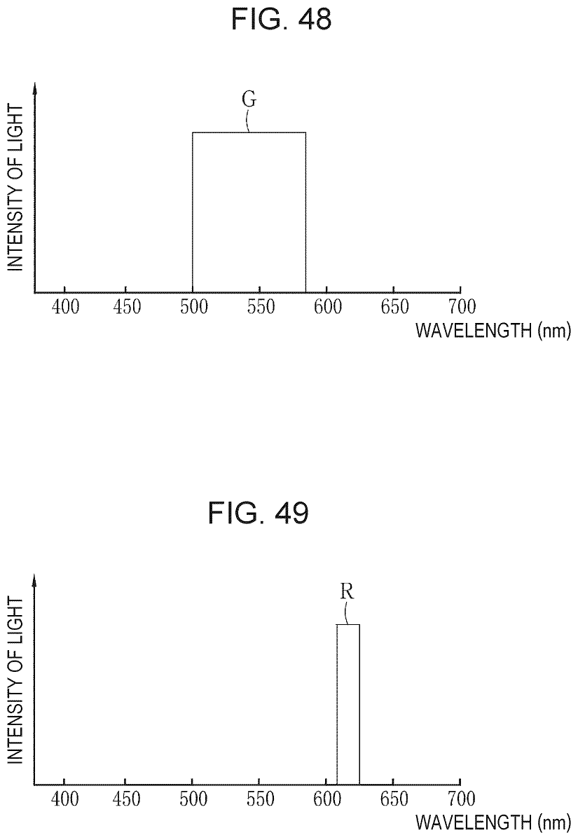

The dichroic mirror 207 reflects, of the light transmitted through the dichroic mirror 206, the green light G and transmits the red light R. As illustrated in FIG. 48, the green light G reflected by the dichroic mirror 207 is incident on the imaging sensor 212. The imaging sensor 212 outputs a Gc image signal in response to the incidence of the green light G in the normal mode, and outputs a G2 image signal in response to the incidence of the green light G in the oxygen saturation mode.

As illustrated in FIG. 49, the red light R transmitted through the dichroic mirror 207 is incident on the imaging sensor 213. The imaging sensor 213 outputs an Rc image signal in response to the incidence of the red light R in the normal mode, and outputs an R2 image signal in response to the incidence of the red light R in the oxygen saturation mode.

### Fourth Embodiment

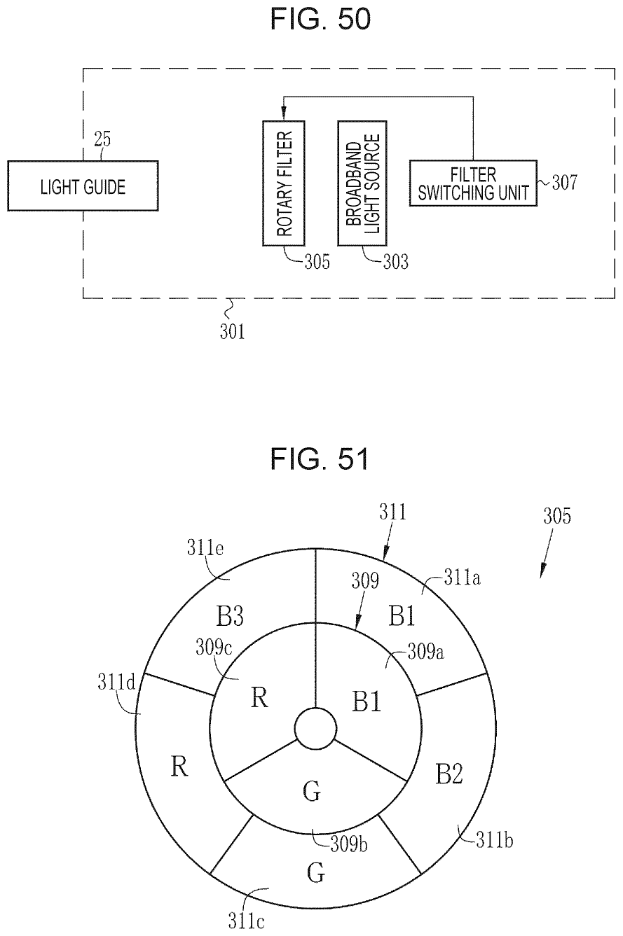

In a fourth embodiment, as illustrated in FIG. 50, in place of the light source device 13 illustrated in the embodiments described above, a light source device 301 including a broadband light source such as a xenon lamp and a rotary filter may be used to illuminate the observation target. In this case, the light source device 301 is provided with a broadband light source 303, a rotary filter 305, and a filter switching unit 307. The broadband light source 303 is a xenon lamp, a white LED, or the like, and emits white light having a wavelength range ranging from blue to red. The imaging optical system is provided with, in place of a color imaging sensor, a monochrome imaging sensor without a color filter. The other elements are similar to those of the embodiments described above, in particular, the first embodiment using the endoscope system 10 illustrated in FIG. 24.

As illustrated in FIG. 51, the rotary filter 305 includes an inner filter 309 disposed on the inner side and an outer filter 311 disposed on the outer side. The filter switching unit 307 is configured to move the rotary filter 305 in the radial direction. When the normal mode is set by the mode switch 12e, the filter switching unit 307 inserts the inner filter 309 of the rotary filter 305 into the optical path of white light. When the oxygen saturation mode is set by the mode switch 12e, the filter switching unit 307 inserts the outer filter 311 of the rotary filter 305 into the optical path of white light.

The inner filter 309 is provided with, in the circumferential direction thereof, a B1 filter 309a that transmits the violet light V and the short-wavelength blue light BS of the white light, a G filter 309b that transmits the green light G of the white light, and an R filter 309c that transmits the red light R of the white light. Accordingly, in the normal mode, as the rotary filter 305 rotates, the observation target is alternately irradiated with the violet light V, the short-wavelength blue light BS, the green light G, and the red light R.

The outer filter 311 is provided with, in the circumferential direction thereof, a B1 filter 311a that transmits the long-wavelength blue light BL of the white light, a B2 filter 311b that transmits the short-wavelength blue light BS of the white light, a G filter 311c that transmits the green light G of the white light, an R filter 311d that transmits the red light R of the white light, and a B3 filter 311e that transmits blue-green light BG having a wavelength range around 500 nm of the white light. Accordingly, in the oxygen saturation mode, as the rotary filter 305 rotates, the observation target is alternately irradiated with the long-wavelength blue light BL, the short-wavelength blue light BS, the green light G, the red light R, and the blue-green light BG.

In the fourth embodiment, in the normal mode, each time the observation target is illuminated with the violet light V, the short-wavelength blue light BS, the green light G, and the red light R, imaging of the observation target is performed by the monochrome imaging sensor. As a result, a Bc image signal, a Gc image signal, and an Rc image signal are obtained. Then, a white-light image is generated on the basis of the image signals of the three colors in a manner similar to that in the first embodiment described above.

In the oxygen saturation mode, by contrast, each time the observation target is illuminated with the long-wavelength blue light BL, the short-wavelength blue light BS, the green light G, the red light R, and the blue-green light BG, imaging of the observation target is performed by the monochrome imaging sensor. As a result, a B1 image signal, a B2 image signal, a G2 image signal, an R2 image signal, and a B3 image signal are obtained. The oxygen saturation mode is performed on the basis of the image signals of the five colors in a manner similar to that of the embodiments described above. In the fourth embodiment, however, a signal ratio ln (B3/G2) is used instead of the signal ratio ln (B3/G3).

In the embodiments described above, the hardware structures of processing units that perform various types of processing, such as the image signal acquisition unit 50, the DSP 51, the noise reducing unit 52, the image processing switching unit 53, the normal image processing unit 54, the oxygen saturation image processing unit 55, the video signal generation unit 56, the correction value setting unit 60, the specific pigment concentration acquisition unit 61, the correction value calculation unit 62, the arithmetic value calculation unit 63, the oxygen saturation calculation unit 64, and the image generation unit 65, are various processors described as follows. The various processors include a CPU (Central Processing Unit), which is a general-purpose processor executing software (program) to function as various processing units, a GPU (Graphical Processing Unit), a programmable logic device (PLD) such as an FPGA (Field Programmable Gate Array), which is a processor whose circuit configuration is changeable after manufacturing, a dedicated electric circuit, which is a processor having a circuit configuration specifically designed to execute various types of processing, and so on.

A single processing unit may be configured as one of these various processors or as a combination of two or more processors of the same type or different types (such as a plurality of FPGAs, a combination of a CPU and an FPGA, or a combination of a CPU and a GPU, for example). Alternatively, a plurality of processing units may be configured as a single processor. Examples of configuring a plurality of processing units as a single processor include, first, a form in which, as typified by a computer such as a client or a server, the single processor is configured as a combination of one or more CPUs and software and the processor functions as the plurality of processing units. The examples include, second, a form in which, as typified by a system on chip (SoC) or the like, a processor is used in which the functions of the entire system including the plurality of processing units are implemented as one IC (Integrated Circuit) chip. As described above, the various processing units are configured by using one or more of the various processors described above as a hardware structure.

More specifically, the hardware structure of these various processors is an electric circuit (circuitry) in which circuit elements such as semiconductor elements are combined. The hardware structure of a storage unit (memory) is a storage device such as an HDD (hard disc drive) or an SSD (solid state drive).

### REFERENCE SIGNS LIST

10 endoscope system
12 endoscope
12a insertion section
12b operation section
12c bending part
12d tip part
12e mode switch
12f still-image acquisition instruction switch
12g tissue-color correction switch
12h zoom operation unit
13 light source device
14 processor device
15 display
16 user interface
17 extension processor device
18 extension display
20 light source unit
20a BS-LED
20b BL-LED
20c G-LED
20d R-LED
20e V-LED
21 light-source processor
23 optical path coupling unit
25 light guide
30 illumination optical system
31 imaging optical system
32 illumination lens
42 objective lens
44 imaging sensor
45 imaging control unit
46 CDS/AGC circuit
48 A/D converter
49 endoscopic operation recognition unit
50 image signal acquisition unit
51 DSP
52 noise reducing unit
53 image processing switching unit
54 normal image processing unit
55 oxygen saturation image processing unit
56 video signal generation unit
57 storage memory
60 correction value setting unit
61 specific pigment concentration acquisition unit
62 correction value calculation unit
63 arithmetic value calculation unit
64 oxygen saturation calculation unit
65 image generation unit
70 curve
71 curve
72 curve
73 100% contour
74 80% contour
75 region
76 region
77 region
81 image display region
81a image display region
81b image display region
81c image display region
81d image display region
82 region of interest
82a region of interest
82b region of interest
82c region of interest
82d region of interest
83 image information display region
84 command region
86a low-reliability region
86b high-reliability region
301 light source device
100 endoscope system
101 endoscope
102 light guide
103 camera head
111 dichroic mirror
115 to 117 image-forming optical system
121 color imaging sensor
122 monochrome imaging sensor
126 solid line
128 broken line
203 camera head
205 to 207 dichroic mirror
210 to 213 imaging sensor
301 light source device
303 broadband light source
305 rotary filter
307 filter switching unit
309 inner filter
309a B1 filter
309b G filter
309c R filter
311 outer filter
311a B1 filter
311b B2 filter
311c G filter
311d R filter
311e B3 filter
B1 image signal
B2 image signal
B3 image signal
BF B color filter
Bk blank frame
BL long-wavelength blue light
BS short-wavelength blue light
CA average specific pigment concentration value
CP specific pigment concentration
CQ specific pigment concentration
CR specific pigment concentration
DFX definition line
DFY definition line
eGr1 effective pixel data
eGr2 effective pixel data
eW1 effective pixel data
eW2 effective pixel data
eW3 effective pixel data
eW4 effective pixel data
G green light
G1 image signal
G2 image signal
G3 image signal
GF G color filter
Gr green frame
Gr1 image signal set
Gr2 image signal set
L low-oxygen region
R red light
R2 image signal
RF R color filter
ROI center region
RX certain range
ST step
V violet light
W white frame
W1 image signal set
W2 image signal set
W3 image signal set
W4 image signal set

What is claimed is:

1. An endoscope system comprising:
a processor configured to:
acquire a first image signal from a first wavelength range having sensitivity to blood hemoglobin;
acquire a second image signal from a second wavelength range different in sensitivity to a specific pigment from the first wavelength range and different in sensitivity to the blood hemoglobin from the first wavelength range;
acquire a third image signal from a third wavelength range having sensitivity to blood concentration;
acquire a fourth image signal from a fourth wavelength range having a longer wavelength than the first wavelength range, the second wavelength range, and the third wavelength range;
receive an instruction to execute a correction value calculation operation for storing a specific pigment concentration from the first image signal, the second image signal, the third image signal, and the fourth image signal, and store the specific pigment concentration by performing the correction value calculation operation a plurality of times;
set a representative value from a plurality of the specific pigment concentrations;
calculate an oxygen saturation, based on an arithmetic value acquired from arithmetic processing using the first image signal, the third image signal, and the fourth image signal and based on the representative value; and
perform an image display using the oxygen saturation.

2. The endoscope system according to claim 1, wherein
the processor is configured to:
have a correlation indicating a relationship between the arithmetic value and the oxygen saturation calculated from the arithmetic value; and
correct the correlation, based on at least the representative value.

3. The endoscope system according to claim 1, wherein
the processor is configured to include a cancellation function of canceling the correction value calculation operation after the correction value calculation operation is performed a plurality of times.

4. The endoscope system according to claim 3, wherein
the processor is configured to implement the cancellation function to delete information on an immediately preceding specific pigment concentration or a plurality of the specific pigment concentrations calculated in the correction value calculation operation.

5. The endoscope system according to claim 1, wherein
the processor is configured to:
store any number of the specific pigment concentrations in the correction value calculation operation in response to a user operation;
terminate the correction value calculation operation in response to the user operation or storage of a certain number of the specific pigment concentrations; and
calculate the representative value when the correction value calculation operation is terminated.

6. The endoscope system according to claim 1, wherein
the processor is configured to:
set a region of interest in an image to be captured, before the correction value calculation operation is performed; and
acquire the specific pigment concentration from an image signal obtained from an image within a range of the region of interest.

7. The endoscope system according to claim 6, wherein
an upper limit number or a lower limit number of the specific pigment concentrations to be stored in the correction value calculation operation varies in accordance with an area of the region of interest, and
the upper limit number of the specific pigment concentrations decreases as the area of the region of interest increases, and the lower limit number of the specific pigment concentrations increases as the area of the region of interest decreases.

8. The endoscope system according to claim 1, wherein
the processor is configured to display information on the specific pigment concentration on a screen when storing the specific pigment concentration.

9. The endoscope system according to claim 1, wherein
the processor is configured to perform the image display such that a region where the oxygen saturation is lower than a specific value is highlighted.

10. The endoscope system according to claim 1, wherein
the specific pigment is a yellow pigment.

11. The endoscope system according to claim 1, further comprising
an endoscope having an imaging sensor provided with a B color filter having a blue transmission range, a G color filter having a green transmission range, and an R color filter having a red transmission range, wherein
the first wavelength range is a wavelength range of light transmitted through the B color filter,
the second wavelength range is a wavelength range of light transmitted through the B color filter,
the second wavelength range is a wavelength range of light having a longer wavelength than the first wavelength range,
the third wavelength range is a wavelength range of light transmitted through the G color filter, and
the fourth wavelength range is a wavelength range of light transmitted through the R color filter.

12. The endoscope system according to claim 11, wherein
the blue transmission range is 380 to 560 nm, the green transmission range is 450 to 630 nm, and the red transmission range is 580 to 760 nm.

13. The endoscope system according to claim 11, wherein
the first wavelength range has a center wavelength of 470±10 nm, the second wavelength range has a center wavelength of 500±10 nm, the third wavelength range has a center wavelength of 540±10 nm, and the fourth wavelength range is a red range.

14. A method for operating an endoscope system, the method comprising:
a step of acquiring a first image signal from a first wavelength range having sensitivity to blood hemoglobin;
a step of acquiring a second image signal from a second wavelength range different in sensitivity to a specific pigment from the first wavelength range and different in sensitivity to the blood hemoglobin from the first wavelength range;
a step of acquiring a third image signal from a third wavelength range having sensitivity to blood concentration;
a step of acquiring a fourth image signal from a fourth wavelength range having a longer wavelength than the first wavelength range, the second wavelength range, and the third wavelength range;
a step of receiving an instruction to execute a correction value calculation operation for storing a specific pigment concentration from the first image signal, the second image signal, the third image signal, and the fourth image signal, and storing the specific pigment concentration by performing the correction value calculation operation a plurality of times;
a step of setting a representative value from a plurality of the specific pigment concentrations;
a step of calculating an oxygen saturation, based on an arithmetic value acquired from arithmetic processing using the first image signal, the third image signal, and the fourth image signal and based on the representative value; and
a step of performing an image display using the oxygen saturation.
